(19) United States
(12) Patent Application Publication
(10) Pub. No.: US 2009/0123447 A1
(43) Pub. Date: May 14, 2009

(54) COMPOSITIONS AND METHODS TO INHIBIT RNA VIRAL REPRODUCTION

(75) Inventor: Jinah CHOI, Merced, CA (US)

Correspondence Address: FOLEY & LARDNER LLP, 975 PAGE MILL ROAD, PALO ALTO, CA, 94304, US

(73) Assignee: The Regents of the University of California

(21) Appl. No.: 12/270,560

(22) Filed: Nov. 13, 2008

Related U.S. Application Data
(60) Provisional application No. 60/988,096, filed on Nov. 14, 2007.

(51) Int. Cl. A61K 31/19; C12N 7/00; A61K 33/40; A61K 38/44; A61P 1/16

(52) U.S. Cl. 424/94.4; 435/235.1; 514/557; 424/616

(57) ABSTRACT

This invention provides methods and compositions for inhibiting replication of the genome of an RNA virus in a host cell by contacting the host cell with an effective amount of an agent that produces a subtoxic concentration of hydrogen peroxide, thereby inhibiting viral replication in the host cell. Also provided are methods and compositions for inhibiting replication of the genome of an RNA virus in a subject in need thereof by administering an effective amount of an agent that produces a subtoxic concentration of hydrogen peroxide in the subject, thereby inhibiting replication. The agents can generate hydrogen peroxide or enhance endogenous levels of hydrogen peroxide. The agents are effective against HCV independent of genotype.

### CROSS-REFERENCE TO RELATED APPLICATIONS

This application claims the benefit under 35 U.S.C. § 119(e) of U.S. Provisional Application Ser. No. 60/988,096, filed Nov. 14, 2007, the contents of which are hereby incorporated by reference in its entirety into the present disclosure.

### BACKGROUND OF THE INVENTION

Throughout this application, patent and technical publications are referenced to more fully describe the state of the art to which this invention pertains. These references are incorporated by reference, in their entirety into this application. The complete bibliographic citation for publications identified by Arabic numerals can be found in this application immediately preceding the claims.

Hepatitis C Virus (HCV) is a positive-sense, single-stranded RNA virus of the Flaviviridae family [9] known to cause serious liver diseases. HCV replication is mediated by NS5B and other nonstructural proteins that comprise the replication complex (RC). HCV infection is associated with increases in various markers of oxidative stress in patients [10], [11], [12] and [13]. Chronic inflammation, iron overload, and some of the HCV proteins may be responsible for some of these changes [10], [14], [15], [16], [17], [18], [19], [20] and [21].

HCV can be transmitted by the blood and is considered to be a common causative agent of post-transfusion non-A, non-B hepatitis (NANBH). An estimated 4.1 million (1.6%) Americans have been infected with HCV, of whom 3.2 million are chronically infected. The risk for perinatal HCV transmission is about 4%. While 80% of infected individuals are often non-symptomatic, the U.S. Center for Disease Control reports that 75 to 85% of HCV infected persons may develop long-term infection, 70% may develop chronic liver disease, 10-20% may develop cirrhosis over a period of 20 to 30 years, and 1-5% of persons may die from the consequences of long term infection (liver cancer or cirrhosis). See: www.cdc.gov/ncidod/diseases/hepatitis/c/fact (last accessed on Nov. 14, 2007).

Hepatitis C is a leading indication for liver transplants. Estimates for the treatment of HCV infection in the United States range form $ 1-5 billion per year; such costs, together with mortality rates, are expected to rise significantly as more and more infected individuals become symptomatic.

A combination of pegylated alpha interferon and ribavirin is the standard treatment for hepatitis C. Alpha interferon is a protein with natural antiviral activity that is made by individuals in response to viral infections. Pegylated interferon, of Peginterferon, is alpha interferon that has been modified chemically by the addition of a large inert molecule of polyethylene glycol in order to change the uptake and distribution and extend half-life. Ribavirin is an oral antiviral agent that by itself has little effect on HCV, but dramatically increases response rate when given in combination with interferon therapy. See, U.S. Patent Publ. No. 20070259844.

Many adverse side effects are associated with therapy (flu-like symptoms, leukopenia, thrombocytopenia, depression, anemia, etc.); only about 50-80% of the patients respond (reduction in serum HCV RNA levels, normalization of liver enzymes); however, of those treated, 50-70% relapse within 6 months of cessation of therapy. Thus, a need exists for safe and effective treatments for HCV infection, replication and diseases incident to infection, are needed. This invention satisfies these needs and provides related advantages as well.

### SUMMARY OF THE INVENTION

This invention provides a method for inhibiting replication of the genome of a retrovirus in a host cell comprising, or alternatively consisting essentially, or yet further consisting of contacting the host cell with an effective amount of an agent that produces a subtoxic concentration of hydrogen peroxide, thereby inhibiting replicating in the host cell. The agents can be administered in a pharmaceutically acceptable carrier. Modes of administration are well known in the art. The agents can generate hydrogen peroxide or enhance endogenous levels of hydrogen peroxide. The agents are effective against HCV independent of genotype.

In another aspect, this invention provides a method for inhibiting replication of the genome of an RNA virus in a subject in need thereof comprising, or alternatively consisting essentially of, or yet further consisting of, administering an effective amount of an agent that produces a subtoxic concentration of hydrogen peroxide in the subject, thereby inhibiting replication. The agents can be administered in a pharmaceutically acceptable carrier. The agents can generate hydrogen peroxide or enhance endogenous levels of hydrogen peroxide. The agents are effective against virus, e.g. RNA viruses such as HCV independent of genotype.

### BRIEF DESCRIPTION OF THE DRAWINGS

FIG. 1. Suppression of HCV RNA by IFN-γ and H₂O₂. SgPC2 cells were treated with various concentrations of H₂O₂ or IFN-γ, as indicated. After 24 h, total RNA was analyzed for HCV RNA and GAPDH mRNA by Northern blots, and the images were analyzed with a phosphoimager (Cyclone; Perkin-Elmer). The intensities of the HCV RNA bands were normalized against that of GAPDH mRNA and expressed as a percentage of the respective control. A representative Northern blot is also shown. Note that these and all other experiments in the subsequent figures were repeated two to eight times, and data are presented as means±SEM of several independent experiments.

FIG. 2 shows buffering [Ca²⁺]ᵢ prevented the suppression of subgenomic and genomic HCV RC by ROS. SgPC2 cells were pretreated with 2 μM BAPTA-AM (Molecular Probes, Inc.) or the vehicle control alone (0.02% DMSO) for 0.5 to 1 h. (A) [Ca²⁺]ᵢ was monitored after loading cells with Indo-1 AM. H₂O₂ (100 μM) was added at 60 s. AUC were 935.9±58.9 Ca²⁺/min (without BAPTA) and 128.4±82.3 Ca²⁺/min (with BAPTA), and the difference was significant p<0.05). (B) SgPC2 cells were labeled with [³H]uridine while treating them with H₂O₂ for 6 h in the presence of actinomycin D. Then, the RNA was isolated and analyzed, as described under Materials and methods. The gel was stained with ethidium bromide to compare the amount of rRNA present in each lane (bottom), which served as the loading control. (C) SgPC2 cells were treated with H₂O₂ for 30 min or 3 h, and the cytoplasmic extracts were subjected to in vitro replication assay. The RNA products were isolated and analyzed on a 1% RNA gel. The gel was stained with ethidium bromide to compare the amount of rRNA in each lane (bottom) as a loading control. (D) SgPC2 cells were treated with H₂O₂ for 24 h. Then, total RNA was analyzed for HCV RNA and GAPDH mRNA by Northern blots, and the images were analyzed with a phosphoimager. The intensities of the HCV RNA bands were normalized against that of GAPDH mRNA and expressed as a percentage of the respective control: H₂O₂, 36.8±16.6% of untreated control; H₂O₂+BAPTA, 139.8±20.2% of BAPTA-treated control. (E) BAPTA removed the suppression of genomic HCV RNA replication by H₂O₂. Huh7 cells were transiently transfected with the genomic HCV RNA and then treated with H₂O₂ for 24 h, with 1 h pretreatment with 2 μM BAPTA-AM or control DMSO. Total RNA was analyzed for HCV RNA by Northern blot. GAPDH mRNA was also analyzed as the control. HCV RNA with H₂O₂ was 34.0±4.0% of untreated control; H₂O₂+BAPTA was 11 1.5±4.5% of BAPTA-treated control.

FIG. 3. Continuous exposure to H₂O₂ suppressed HCV RNA replication. SgPC2 cells were treated with GO for 24 h, with and without cotreatment with 130-200 U/ml catalase or with 1 h pretreatment with 2 μM BAPTA-AM or DMSO. (A and B) HCV RNA and GAPDH mRNA levels were determined by Northern blot analysis and the images were analyzed with a phosphoimager. The intensities of the HCV RNA bands were normalized against that of GAPDH mRNA and expressed as a percentage of the control. Data represent means±SEM. (C) HCV NS5A protein level was analyzed by Western blot, as described under Materials and methods.

FIG. 4. GSH and HCV RNA replication. SgPC2 cells were incubated with 20-40 μM BSO with and without H₂O₂ for 24 h and analyzed for (A) GSH or (B) HCV RNA and GAPDH mRNA levels by Northern blots. Total GSH was expressed as nmol/mg total protein. The RNA bands were analyzed with a phosphoimager. GAPDH mRNA served as the control. Data represent means±SEM. *Statistically significant difference by Student's t test (p≦0.05).

FIG. 5. H₂O₂ induced calcium release from the ER. SgPC2 cells were loaded with Indo-1 AM, and [Ca²⁺]ᵢ was monitored in the absence of extracellular calcium, unless specified otherwise. (A) H₂O₂ or water was added at 60 s and calcium was monitored in the presence and absence of 1.3 mM extracellular calcium. AUC of peroxide-induced calcium elevations were 1278.5±109.5 Ca²⁺/min, with extracellular calcium, and 1105.1±238.1 Ca²⁺/min, without extracellular calcium, which were not different (p>0.05). (B and C) Cells were pretreated with (B) H₂O₂ for 13 min or (C) 400 nM TG or control DMSO for 10 min before calcium measurement. Arrows indicate the addition of peroxide (A and C) or TG (B). H₂O₂ decreased TG-induced calcium elevation to 9.2±2.6% of control (AUC of TG-induced calcium response in control cells, 772.3±189.3 Ca²⁺/min; p<0.05) (B). Likewise, TG decreased the peroxide-induced calcium elevation to 58.3±8.4% of the control (p<0.05) (C).

FIG. 6. The suppression of HCV replication by H₂O₂ required an internal calcium store(s). SgPC2 cells were incubated in normal medium (10% FBS) or medium with 0% FBS, serum-starved overnight with 0.5% FBS and then incubated in 0% FBS for 1 h, incubated in KRPH, or pretreated with DMSO control or 5 μM ionomycin in calcium-free KRPH buffer for 15 min. Then, the cells were treated with H₂O₂ for 30 min. The cytoplasmic lysates were prepared and analyzed for in vitro HCV replication. The rRNA gel shows RNA loading. HCV RNA bands were analyzed with a phosphoimager, normalized against the rRNA bands, and expressed as a percentage of the respective control. Data represent means±SEM.

FIG. 7. Elevated calcium might be sufficient to suppress HCV replication. (A) SgPC2 cells were treated with 400 nM and 2 μM TG, DMSO control, H₂O₂, or 10 μM ionomycin for 15 min. Then, cytoplasmic lysates were prepared and analyzed for in vitro HCV replication. Data represent means±SEM. (B) CaCl₂ was added to untreated cytoplasmic lysates at concentrations shown before in vitro replication assay. The rRNA gel shows RNA loading. The RNA bands were analyzed with a phosphoimager, normalized against the rRNA bands, and expressed as a percentage of the respective control. Data represent means±SEM. (C) CaCl₂ was added to purified full-length NS5B at concentrations shown before in vitro RdRp assay. ZnCl₂ (0.5 mM) was used as a positive control.

FIG. 8. H₂O₂ suppresses JFH1 HCV RNA levels. (A) JFH1-transfected cells were exposed to glucose oxidase plus glucose (with and without BSO) (A) or bolus addition of H₂O₂ (B—C), and analyzed for HCV RNA levels by qRT-PCR. Enzymatic production of H₂O₂ by glucose oxidase+glucose decreases HCV RNA in JFH1-transfected Huh7 cells and exacerbates the decrease caused by BSO alone. Bolus H₂O₂ decreases both intracellular (B) and extracellular (C) JFH1 HCV RNA.

FIG. 9. BSO decreases HCV RNA levels by depleting intracellular GSH. Intracellular (A) and extracellular (B) JFH1 HCV RNA were extracted and analyzed by Real time qRT-PCR after 24 hr treatment with BSO, with and without additional 24 hr. treatment with GSH or GSH ester. Only GSH ester is able to restore HCV RNA level, decreased with BSO

FIG. 10. H₂O₂ suppresses HCV RNA genome replication. Huh7 cells were transfected with SgJFH1-Luc RNA and then treated with glucose oxidase plus glucose for 24 hrs with or without pretreatment with BSO. Then, the cell lysates were analyzed for luciferase activity.

FIG. 11. DPI enhances HCV RNA level. Huh7 cells supporting continuous replication of HCV Con1 subgenomic RNA were treated with DPI and analyzed for HCV and GAPDH RNA's by northern blot.

### DETAILED DESCRIPTION OF THE INVENTION

### Abbreviations

The use of the following abbreviations facilitate the description of the disclosed inventions:

BAPTA-AM, 1,2-bis(2-aminophenoxy)ethane-N,N,N′-tetraacetic acid tetrakis acetoxymethyl ester; BSO, 1-buthionine S,R-sulfoximine; [Ca^{2+]}ᵢ, intracellular calcium concentration; DMEM, Dulbecco's modified Eagle medium; DMSO, dimethyl sulfoxide; EGTA, ethylene glycol-bis(β-aminoethyl)-N,N,N′,N′-tetraacetic acid; ER, endoplasmic reticulum; FBS, fetal bovine serum; GAPDH, glyceraldehyde-3-phosphate dehydrogenase; GO, glucose oxidase; GSH, glutathione; HCV, hepatitis C virus; IFN-γ, interferon-γ; IP₃, inositol 1,4,5-triphosphate; KRPH, Krebs-Ringer phosphate buffer; NAC, N-acetylcysteine; NF-κB, nuclear factor KB; RC, replication complexes; ROS, reactive oxygen species; TG, thapsigargin.

### DEFINITIONS

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N. Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

HCV is a member of the Flavivirus family. Others include, but are not limited to GB virus B, Japanese Encephalovirus (JEV) and West Nile Virus (WNV).

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype” refers to the detectable outward manifestations of a specific genotype.

A “probe” when used in the context of polynucleotide manipulation refers to an oligonucleotide that is provided as a reagent to detect a target potentially present in a sample of interest by hybridizing with the target. Usually, a probe will comprise a detectable label or a means by which a label can be attached, either before or subsequent to the hybridization reaction. Alternatively, a “probe” can be a biological compound such as a polypeptide, antibody, or fragments thereof that is capable of binding to the target potentially present in a sample of interest.

“Detectable labels” include, but are not limited to radioisotopes, fluorochromes, chemiluminescent compounds, dyes, and proteins, including enzymes. Detectable labels can also be attached to a polynucleotide, polypeptide, antibody or composition described herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as “replication.” A primer can also be used as a probe in hybridization reactions, such as Southern or Northern blot analyses. Sambrook and Russell (2001), infra.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

“Hydrogen peroxide” is intended to mean H₂O₂, and is intended encompass a common precourser “superoxide” and other reactive oxygen species.

“Glucose Oxidase” is intended to mean an enzyme which catalyzes the oxidation of β-D-glucose to D-glucono-1,5-lactone and hydrogen peroxide using molecular oxygen as the electron acceptor. Glucose oxidase can be purchased from commercial sources such as Sigma. Other enzymes known to produce hydrogen peroxide are cholesterol oxidase, urea oxidase and human monocytes.

“L-buthionine-S,R-sulfoximine” or “BSO” or “L-Buthionine-sulfoximine” is intended to mean L-buthionine-S,R-sulfoximine, D-buthionine-S,R-sulfoximine, or a mixture thereof. BSO can be purchased from commercial sources such as Sigma.

“Ascorbate” is intended to mean 2-oxo-L-threo-hexono-1,4-lactone-2,3-enediol, (R)-3,4-dihydroxy-5-((S)-1,2-dihydroxyethyl)furan-2(5H)-one, ascorbic acid or vitamin-C or the ionized form thereof “Dehydroascorbate” is intended to mean dehydroascorbic acid (DHA) or the ionized form thereof, or an oxidized form of ascorbate.

“NAD(P)H oxidase” is intended to mean nicotinamide adenine dinucleotide phosphate-oxidase. NAD(P)H oxidase is or “NOx Protiens” Suitable compounds for inclusion in the methods of this invention include, for example, other sources of reactive oxygen species include the NADPH oxidases, xanthine oxidase, uncoupled nitric oxide synthase, and mitochondrial sources

“BCNU” is intended to mean 1,3-bis(chloroethyl)-1-nitrosourea. BCNU, also known as Carmustine, is used as an alkylating agent in chemotherapy. Carmustine for injection is marketed under the name BiCNU by Bristol-Myers Squibb.

“Quinone” is intended to mean a cyclohexadienedione compound or derivative thereof. Derivatives of such compounds include, but are not limited to tert-butylhydroquinone (TBHQ). These compounds are commercially available from sources such as Sigma. Suitable compounds for inclusion in the methods of this invention include, for example, 1,4-naphthoquinone, 5-hydroxy-1,4-naphthoquinone, 2-hydroxy-1,4-naphthoquinone, 2-methyl-1,4-naphthoquinone (menadione), 5-hydroxy-2-methyl-1,4-naphthoquinone, 3-hydroxy-2-methyl-1,4-naphthoquinone, 2,3-dimethyl-1,4-naphthoquinone and 2,3-dimethoxy-1,4-naphthoquinone (DMNQ). Suitable compounds for inclusion in the methods of this invention also include quinone anticancer agents, for example, diazyquone (AZQ), andriamycin, 2,5-diaziridinyl-1,4-benzoquinone (DZQ), and derivatives thereof.

“Butylated hydroxyanisole” or “BHA” is intended to mean a mixture of 2-tert-butyl-4-hydroxyanisole and 3-tert-butyl-4-hydroxyanisole. Suitable compounds for inclusion in the methods of this invention include, for example, other butylated phenols such as butylated hydroxytoluene (BHT). BHA and BHT can be purchased from Sigma.

A “composition” is intended to mean a combination of active polypeptide, polynucleotide or antibody and another compound or composition, inert (e.g. a detectable label) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which is susceptible to RNA viral infection. In one embodiment, the mammals include horses, dogs, and cats. In another embodiment of the present invention, the human is an adolescent or infant under the age of eighteen years of age.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The terms “disease,” “disorder,” and “condition” are used inclusively and refer to any condition mediated at least in part by infection by an RNA virus such as HCV.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication in vitro or in vivo.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

### Methods to Inhibit RNA Virus Replication

This invention provides a method for inhibiting replication of the genome of a virus such as an RNA virus in a host cell by contacting the host cell with an effective amount of an agent that produces a subtoxic concentration of hydrogen peroxide, thereby inhibiting replication in the host cell. The agents can generate hydrogen peroxide or enhance endogenous levels of hydrogen peroxide. The agents are effective against HCV independent of genotype.

As used herein, any suitable cell that supports RNA viral reproduction and genomic replication is suitable for this method. Examples of such include, eukaryotic cells such as animals, e.g., murines, rats, rabbit, simians, bovines, ovine, porcine, canines, feline, farm animals, sport animals, pets, equine, and primate, particularly human. The cells can be cultured cells or they can be primary cells. Cultured cell lines can be purchased from vendors such as the American Type Culture Collection (ATCC), U.S.A. In one particular embodiment, the cells are liver cells, e.g., cultured liver cells or primary liver cells. The cells are infected with RNA virus and may further exhibit pathology such as a liver carcinoma.

Suitable agents for use in this method include, for example, an agent from the group enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)-a redox cycling quinine, 2,3 dimethoxy-1,4-naphthoquinone (DMNQ)-a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

In one aspect, the agent is BSO. In another aspect, the agent is a combination of an effective amount of hydrogen peroxide and L-buthionine S,R-sufloximine (BSO). In a further aspect, the agent is hydrogen peroxide alone or in combination with BSO. In another aspect, the agent is a combination of glucose and glucose oxidase. The agents, alone or in combination, can be formulated into pharmaceutical compositions or they can be directly contacted with the cell.

Suitable examples of RNA viruses that are inhibited by these methods include, but are not limited to, Flavivirus, (e.g., HCV). Other viruses that may be affected similarly will include Dengue virus, yellow fever virus and West Nile Virus. Another virus known to be inhibited by hydrogen peroxide is hepatitis B virus, a DNA virus that also infects and damages liver.

In one aspect of the above methods, the agent increases endogenous reactive oxygen species (ROS). Methods to determine endogenous ROS are known in the art and described in the experimental section below. Methods to determine if RNA viral replication has been reduced or inhibited also are known in the art and briefly described herein.

The method can also be practiced by contacting with an agent that produces mild endogenous oxidative stress. In an alternate embodiment, the agent reduces intracellular glutathione.

The methods are useful to inhibit the replication of RNA viruses. Examples of positive-sense stranded RNA viruses include, but are not limited to Flavivirus, e.g., HCV.

The methods of this invention present unexpected advantage by inhibiting or reducing subgenomic viral replication indicating that RNA genome replication is inhibited. Methods to determine subgenomic viral replication are known in the art and briefly described herein. An additional unexpected advantage is that the methods inhibit the complete viral replication cycle. Methods by inhibiting intracellular genome replication to determine if the complete retroviral life cycle have been completed in the art and briefly described herein.

The methods can be practiced in vitro or in vivo. When practiced in vitro, they are effective means to identify and test therapeutic agents and regimens before advancement into the clinic. By having two cell systems, one can test or screen a potential therapeutic and compare its activity to those agents and combinations described herein. The methods can be modified for high-throughput testing of agents and potential therapeutics.

In vivo practice of the invention in an animal such as a rat or mouse provides a convenient animal model system that can be used prior to clinical testing of the agent. In this system, a potential agent, compound or composition will be successful if retroviral replication is reduced or the symptoms of the infection are ameliorated as compared to an untreated, infected animal. It also can be useful to have a separate negative control group of cells or animals which has not been infected, which provides a basis for comparison.

When practiced in vivo, the candidate prodrug is administered or delivered to the animal in effective amounts. As used herein, the term “administering” for in vivo and ex vivo purposes means providing the subject with an effective amount of the candidate agent effective to inhibit retroviral replication as described herein. In these instances, the agent, compound or composition may be administered with a pharmaceutically acceptable carrier. These agents and combinations also can be used in the manufacture of medicaments and for the treatment of humans and other animals by administration in accordance with conventional procedures, such as an active ingredient in pharmaceutical compositions.

This invention provides a method for inhibiting replication of the genome of an RNA virus in a subject in need thereof by administering an effective amount of an agent that produces a subtoxic concentration of hydrogen peroxide, thereby inhibiting replication in the host cell. The agents can generate hydrogen peroxide or enhance endogenous levels of hydrogen peroxide. The agents are effective against HCV independent of genotype.

A subject in need thereof may be animals, e.g., murines, rats, rabbit, simians, bovines, ovine, porcine, canines, feline, farm animals, sport animals, pets, equine, and primate, particularly human.

Suitable agents for use in this method include, for example, an agent from the group enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)-a redox cycling quinine, 2,3 dimethoxy-1,4-naphthoquinone (DMNQ)-a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

In one aspect, the agent is BSO. In another aspect, the agent is a combination of an effective amount of hydrogen peroxide and L-buthionine S,R-sufloximine (BSO). In a further aspect, the agent is hydrogen peroxide alone or in combination with BSO. In another aspect, the agent is a combination of glucose and glucose oxidase. The agents, alone or in combination, can be formulated into pharmaceutical compositions or they can be directly contacted with the cell.

The agents, alone or in combination, can be formulated into pharmaceutical compositions or they can be directly contacted with the cell.

The methods are useful to inhibit the replication of an RNA virus. Suitable examples of RNA viruses which infect humans include, but are not limited to Flavivirus, e.g., HCV. Other viruses that may be affected similarly will include Dengue virus, yellow fever virus and West Nile Virus. Another virus known to be inhibited by hydrogen peroxide is hepatitis B virus, a DNA virus that also infects and damages the liver.

The methods of this invention present unexpected advantage by inhibiting or reducing subgenomic viral replication without virus production. Methods to determine subgenomic viral replication are known in the art and briefly described herein. An additional unexpected advantage is that the methods inhibit the complete retroviral replication cycle. Methods to determine if the complete retroviral life cycle has been completed are known in the art and briefly described herein.

Also provided herein is a method for treating diseases incident to RNA viral infection, e.g., liver disease incident to Hepatitis C Viral infection, in a subject by administering to the subject an effective amount of an agent that produces a subtoxic concentration of hydrogen peroxide in the subject. The agents can generate hydrogen peroxide or enhance endogenous levels of hydrogen peroxide. The agents are effective against HCV independent of genotype.

A subject in need thereof may be animals, e.g., murines, rats, rabbit, simians, bovines, ovine, porcine, canines, feline, farm animals, sport animals, pets, equine, and primate, particularly human.

Suitable agents for use in this method include, for example, an agent from the group of enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)-a redox cycling quinine, 2,3 dimethoxy-1,4-naphthoquinone (DMNQ)-a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

In one aspect, the agent is BSO. In another aspect, the agent is a combination of an effective amount of hydrogen peroxide and L-buthionine S,R-sufloximine (BSO). In a further aspect, the agent is hydrogen peroxide alone or in combination with BSO. In another aspect, the agent is a combination of glucose and glucose oxidase. The agents, alone or in combination, can be formulated into pharmaceutical compositions or they can be directly contacted with the cell.

Examples of liver disease incident to HCV include, but are not limited to cirrhosis, steatosis or hepatocellular carcinoma.

Subjects that are suitably treated are described above and include any animal, vertebrate or mammal that is susceptible to RNA viral, and for example, HCV infection. Persons at risk for HCV infection include injecting drug users, recipients of clotting factors made before 1987, hemodialysis patients, recipients of blood and/or solid organs before 1992, people with undiagnosed liver problems, infants born to infected mothers and healthcare/public safety workers after known exposure.

### Compositions

This invention also provides compositions containing the active agent as described herein to inhibit RNA viral replication. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

An “effective amount” of the agent or composition is contacted with the cell, in vitro or can be administered to the subject such as a human patient, in vivo. An effective amount is an amount sufficient to effect beneficial or desired results. An effective amount can be administered in one or more administrations, applications or dosages.

The invention provides an article of manufacture, comprising packaging material and at least one vial comprising a solution of at least one agent or composition with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising at least one agent or composition and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

In some aspects, the agent or composition is prepared to a concentration includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

The formulations of the present invention can be prepared by a process which comprises mixing at least one agent or composition and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of agent or composition that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include pen-injector devices for delivery of a solution such as BD Pens, BD Autojectore, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a therapeutic agent of the invention, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the invention locally to the area in need of treatment; this may be achieved by, for example, and not by way of limitation, local infusion during surgery, by injection or by means of a catheter.

Solutions containing the agent(s) can be prepared in suitable diluents such as water, ethanol, glycerol, liquid polyethylene glycol(s), various oils, and/or mixtures thereof, and others known to those skilled in the art.

The pharmaceutical forms of the agent(s) suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

The following examples are intended to illustrate but not limit the invention.

### EXPERIMENTAL EXAMPLES

### Example 1

Reactive oxygen species (ROS) and other reactive species are products of normal cell metabolism [1]. The synthesis of reactive species, however, is heightened during inflammation [2]. The oxidative stress that ensues is believed to help fight off various infections, for example, by inflicting oxidative injury to the invading pathogens. In fact, increased levels of reactive species and decreased levels of antioxidant molecules have now been documented in many viral diseases. However, ROS also affect and participate in signaling [3], [4] and [5] and, in this manner, may have other effects on viruses. For example, ROS can negatively regulate hepatitis B virus replication in liver cells without affecting the cell metabolism [6] but enhance the replication of human immunodeficiency virus by activating nuclear factor KB (NF-κB) [7]. Likewise, sublethal and biologically relevant concentrations of ROS and, in particular, H₂O₂ have been found to rapidly suppress hepatitis C virus (HCV) RNA replication in Huh7 human hepatoma cells in a manner that suggested signaling [8].

HCV is a positive-sense, single-stranded RNA virus of the Flaviviridae family [9]. HCV replication is mediated by NS5B and other nonstructural proteins that comprise the replication complex (RC). HCV infection is associated with increases in various markers of oxidative stress in patients [10], [11], [12] and [13]. Chronic inflammation, iron overload, and some of the HCV proteins may be responsible for some of these changes [10], [14], [15], [16], [17], [18], [19], [20] and [21]. Increased levels of ROS and nitrogen species are suggested to enhance the pathogenesis of HCV by promoting DNA damage and steatosis [19] and [22]. In addition, H₂O₂ has been found to suppress HCV RNA replication in cell culture [8]. This suppression was accompanied by a loss of HCV proteins and HCV replicating activity that cofractionated with the Golgi membranes. There was no change in host cell viability, overall intracellular redox status, housekeeping-gene expression, ribosomal RNA synthesis, or subcellular distribution of albumin with the peroxide treatment, suggesting that the suppression was rather specific to HCV.

Therefore, in this study, whether ROS rapidly suppressed the activity of the HCV replication complex through redox signaling was tested to understand the biological consequence of increased oxidative stress in hepatitis C. The effect of H₂O₂ on HCV RNA replication is also compared with that of interferon-γ (IFN-γ). Interestingly, various HCV proteins have been suggested to modulate calcium metabolism [17], [23] and [24]. HCV p7 protein may act as a calcium channel [24]. As sublethal concentrations of oxidants can initiate calcium signaling [25], [26], [27] and [28], we examined the possible involvement of calcium in the modulation of the HCV RC by H₂O₂. Our data indicate that H₂O₂ causes a gradual rise in the intracellular calcium concentration ([Ca²⁺]ᵢ) in Huh7 cells, at least in part, by releasing calcium from the endoplasmic reticulum. Buffering this calcium completely removes the oxidative suppression of the RC. Other agents that elevate intracellular calcium concentration can elicit a similar suppressive response. These data highlight the importance of redox and calcium homeostasis during HCV infection. Possible implications of these findings are discussed.

### Materials and Methods

### Cell Culture and Electroporation of HCV RNA:

Huh7 human hepatoma cells were cultured in Dulbecco's modified Eagle medium (DMEM; Invitrogen, Carlsbad, Calif., USA) supplemented with 10% fetal bovine serum (FBS; Invitrogen), 100 units/ml of penicillin, and 100 μg/ml of streptomycin in a 5% CO₂ incubator. SgPC2 cells are G418-selected, pooled Huh7 cell clones that support the continuous replication of a subgenomic HCV replicon of genotype 1b [8] and [29] (GenBank Accession No. AJ242652) that carries a S11791 adaptive mutation within the NS5A region [30]. SgPC2 cells were maintained in the above medium supplemented with 0.5 mg/ml G418 (Invitrogen). G418 was removed at least 1 day before cell treatments.

For transient replication experiments, the hybrid genomic plasmid [8] was linearized with XbaI and used to synthesize genomic HCV RNA, as previously described [8]. Then, 5×10⁶ Huh7 cells were rinsed with DMEM, mixed briefly with 10-20 μg of the subgenomic replicon RNA in 0.4 ml of DMEM, and then electroporated at 220 V and 975 μF. Then, 1 million cells were seeded onto 60-mm cell culture dishes in DMEM containing 10% FBS. Medium was changed daily and, after 2-4 days, the cells were preincubated with 1,2-bis(2-aminophenoxy)ethane-N,N,N′,N′-tetraacetic acid tetrakis acetoxymethyl ester (BAPTA-AM) or vehicle control alone for 1 h. Then, the cells were exposed to H₂O₂ for 24 h, and the amount of the HCV RNA was analyzed by Northern blot (see below). A negative control RNA (H77c) [30] and [31] has been shown not to replicate under these conditions [8].

### [Ca²⁺]₁ Measurement

Huh7 and SgPC2 cells were grown as monolayers on glass coverslips, and [Ca²⁺]ᵢ was monitored as previously described [32]. Briefly, on the day of the experiment, cells were washed in Krebs-Ringer phosphate buffer (KRPH; pH 7.4, containing 1.0 mM MgSO₄, 1.3 mM CaCl₂, 10 mM Hepes, 5 mM glucose, 125 mM NaCl, 5 mM KCl, and 10 mM sodium phosphate) and subsequently loaded with 10-20 μM Indo-1 AM (70 μl/coverslip; Molecular Probes, Inc., Eugene, Oreg., USA) containing 10 μM probenecid (Molecular Probes) for 30 min at 37° C. The attached cells were washed and bathed in KRPH/probenecid at 4° C. or room temperature until further analysis. Then, coverslips were individually submerged into KRPH and preequilibrated inside the F2000 fluorimeter (Hitachi) in darkness for 3 min, and [Ca²⁺]ᵢ was monitored continuously at 37° C., with excitation at 380 nm and emission at 400 and 450 nm. H₂O₂ and other agents were injected at different time points, as indicated under Results. [Ca²⁺]ᵢ was calculated as described [32]. Rₘₐₓ was determined by adding 8-16 μM digitonin and Rₘᵢₙ with 10 mM ethylene glycol-bis(β-aminoethyl)-N,N,N′-tetraacetic acid (EGTA). Adding 10-fold higher concentration of digitonin did not alter the Rₘₐₓ. The baseline, which represented unstimulated cells scanned over time, was plotted along with other measurements or subtracted from the other plots. Cells were serum-depleted, 1 day before the experiment, by incubating them overnight in DMEM plus 0.5% FBS. The serum depletion or the absence of serum in the cell incubation buffer did not affect the suppression of HCV replication in SgPC2 cells by H₂O₂ (see Results). For studies that required the absence of extracellular calcium, KRPH was prepared without CaCl₂, and 0.1 mM EGTA was added. Then, CaCl₂ was added before determining the Rₘₐₓ for each run. Areas under the curve (AUC) were calculated, using the “area below the curve” algorithm of SigmaPlot 9.0 (Jandel Scientific).

### Enzymatic Generation of H₂O₂ by Glucose Oxidase (GO)

Cells were incubated with various concentrations of GO (Sigma-Aldrich, St. Louis, Mo., USA) in the cell culture medium (DMEM, high glucose-already contains 25 mM β-d(+)-glucose, which is needed for the reaction), supplemented with 10% fetal bovine serum, for 24 h [33]. Then, the cells were washed with phosphate-buffered saline and harvested as indicated under Results. To confirm the role of exogenous H₂O₂ in the modulation of HCV replication by GO, 130 to 200 U/ml catalase (Sigma-Aldrich) was added.

### ³H Labeling of Newly Synthesized HCV RNA

One million SgPC2 cells were pretreated with 2 μM BAPTA-AM or DMSO alone for 1 h and then treated with 0 or 100 μM H₂O₂ in the presence of 100 μCi of [³H]uridine (35-50 Ci/mmol; MP Biomedicals, Irvine, Calif., USA) and 4 μg/ml actinomycin D (Sigma-Aldrich) for 5 to 6 h and then lysed for the RNA isolation. RNA was analyzed on a 1% formaldehyde agarose gel, which was then treated with 1 M sodium salicylate for 20-30 min for fluorography. The RNA gel was also stained with ethidium bromide to visualize rRNA bands to serve as loading controls.

### Northern Blot Analysis

Total RNA was extracted from cells, using Trizol reagent (Invitrogen), following the manufacturer's protocol. Five to 20 μg of RNA was then subjected to Northern blot analysis using a ³²P-labeled double-stranded DNA probe prepared from nt 3669 to nt 6016 of the subgenomic replicon. For these and all other experiments, RNA and protein bands were quantified with a phosphoimager (Cyclone; Perkin-Elmer) or by densitometry using SigmaScan (Jandel Scientific) or IS2000R (Kodak). The intensities of the bands of interest were normalized against the control glyceraldehyde-3-phosphate dehydrogenase (GAPDH) mRNA.

### Western Blot Analysis of HCV NS5A

Cells were sonicated in 2× Laemmli buffer and total protein samples were analyzed for HCV NS5A level by Western blot, using monoclonal anti-NS5A antibodies (Biodesign) and the corresponding horseradish peroxidase-conjugated secondary antibodies. Chemiluminescent images were processed with an ECL kit from Amersham Pharmacia and analyzed with the IS2000 gel imaging system (Kodak).

### In Vitro HCV Replication Assay

In vitro replication assay was carried out according to the protocol of Ali et al. [34], with slight modifications. Briefly, SgPC2 cells were grown to approximately 50-60% confluence in 100-mm dishes. Then, the cells were treated for 30 min with various agents, and cytoplasmic lysates were prepared by gently lysing the cells in the incomplete replication buffer (prepared without dithiothreitol) immediately after treating the cells with lysolecithin-containing washing solution (150 mM sucrose, 30 mM Hepes, pH 7.4, 33 mM ammonium chloride, 7 mM KCl, 4.5 mM magnesium acetate, 250 μg/ml lysolecithin) for 1-2 min. Twenty to 50 μCi of [α-³²P]CTP (3000 Ci/mmol) and 1-2 μg of actinomycin D were added to 70 μl of these cytoplasmic lysates, and the replication reaction was allowed to proceed for 1 h at 30° C. Then, RNA was isolated by phenol:chloroform extraction and ethanol precipitation. The ³²P-labeled transcripts were then analyzed on a 1% formaldehyde agarose gel which was subsequently washed with H₂O, dried, and analyzed with a phosphoimager.

### Determination of Intracellular Glutathione (GSH) and ATP Concentrations

Intracellular GSH concentration was determined using a GSH recycling assay [35] and expressed as nmol/mg protein. ATP level was measured using the Somatic Cell ATP assay kit from Sigma-Aldrich, as previously described [32]. Protein level was determined with a bicinchoninic acid kit (Pierce Biotechnology, Inc., Rockford, Ill., USA).

### Mitochondrial Membrane Potential and Apoptosis

Mitochondrial membrane potential was measured with JC1, using a Guava cytometer (Guava Technologies, Inc.). Apoptosis was also monitored with annexin V-FITC, with a 96-Nexin kit from Guava Technologies, Inc.

### NS5B RdRp Assay

Full-length NS5B protein was purified from Escherichia coli, as previously described, and was used to catalyze de novo RNA synthesis in vitro [36].

### Statistics

Data are presented as means±SEM (standard error of the mean) of several independent experiments. Data were analyzed using Student's t test or one-way analysis of variance. A p value ≦0.05 was considered significant. All experiments were repeated two to eight times.

### Results

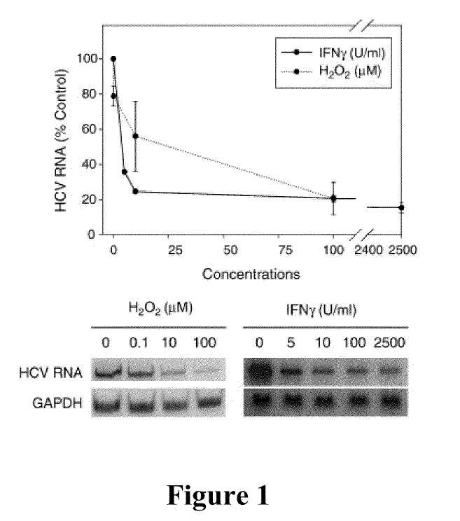

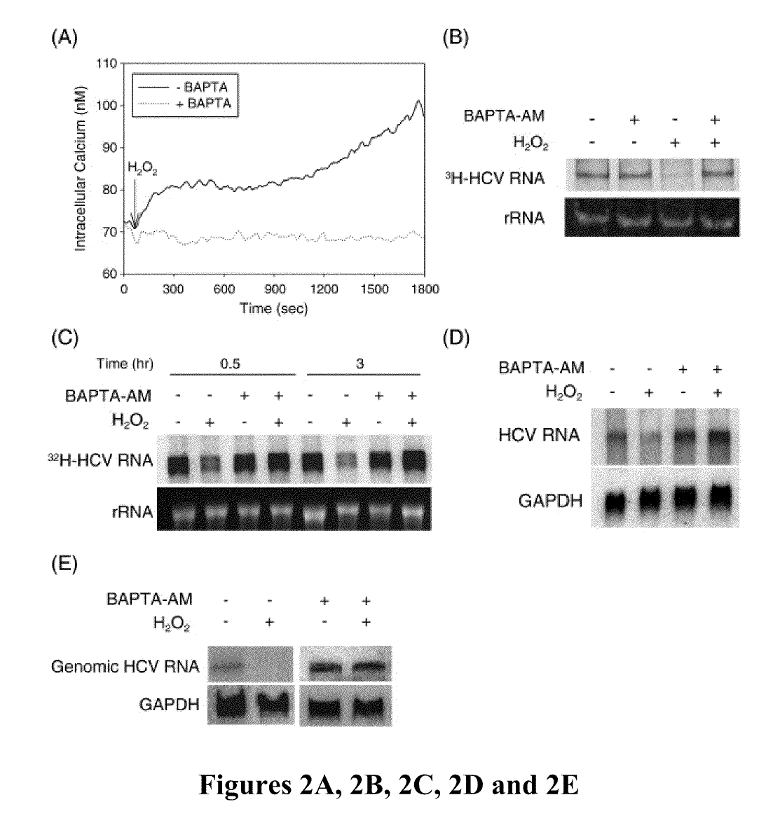

First, we compared the HCV RNA-lowering activity of H₂O₂ with that of interferon-γ (IFN-γ), which is known to inhibit HCV RNA replication in Huh7 human hepatoma cells [37]. Huh7-derived SgPC2 cells, which support the continuous replication of Con1 subgenomic HCV RNA [8], [29] and [30], were incubated with H₂O₂ or IFN-γ for 24 h and analyzed for HCV RNA level by Northern blots. As shown in FIG. 1, IFN-γ decreased the HCV RNA level dose dependently, to about 15% of control level at the concentration of 100 U/ml. H₂O₂ also suppressed HCV RNA level dose dependently, with an IC₅₀ of about 25 μM, and 100 μM H₂O₂ was as effective as 100 to 2500 U/ml IFN-γ at suppressing the HCV RNA level (FIG. 1). Higher concentrations of H₂O₂ were not tested, to stay within the subtoxic range. Unlike H₂O₂, which showed a significant suppression of HCV RNA replication within 30 min of treatment [8] (also, see FIG. 2), IFN-γ treatment had no appreciable effect on HCV RNA replication after 30 min (data not shown). Therefore, H₂O₂ showed a faster onset of suppressive activity than IFN-γ.

H₂O₂ has been shown to elevate intracellular calcium concentration in primary hepatocytes [27] and [38]. To elucidate the mechanism underlying the suppression of HCV replication by H₂O₂, we examined whether H₂O₂ induces calcium elevation in SgPC2 cells. SgPC2 cells were loaded with Indo-1 AM and monitored for changes in [Ca²⁺]ᵢ. H₂O₂ caused a gradual rise in [Ca²⁺]ᵢ (FIG. 2A) that was dose dependent and persisted for at least 2 h (data not shown). The calcium response of SgPC2 cells was comparable to that of naïve Huh7 cells (data not shown).

To test whether this elevation of [Ca²⁺]ᵢ played a role in the oxidative suppression of HCV replication, we used BAPTA-AM, which effectively buffered [Ca²⁺]ᵢ in these cells (FIG. 2A). Then, SgPC2 cells, pretreated with either BAPTA-AM or control vehicle alone, were incubated with [³H]uridine and actinomycin D, with or without H₂O₂, for 6 h, to look for changes in the rate of appearance of newly synthesized HCV transcripts. H₂O₂ significantly decreased the amount of the HCV RNA that was synthesized during these 6 h (FIG. 2B). Interestingly, BAPTA-AM had no significant effect on the HCV RNA replication by itself but completely removed the suppression of HCV RNA replication by H₂O₂ (FIG. 2B).

To determine the time course of calcium dependence of this suppression by H₂O₂, control and BAPTA-pretreated cells were exposed to H₂O₂ for 30 min or 3 h, and the cytoplasmic lysates that contained HCV RC were analyzed for in vitro HCV RNA replication. H₂O₂ treatment led to the suppression of in vitro HCV replication (FIG. 2C). BAPTA-AM removed this suppression at both time points tested (FIG. 2C), although the protective effect of BAPTA-AM was more variable at 30 min. Similarly, buffering [Ca²⁺]ᵢ inhibited the peroxide-induced decrease in the total HCV RNA level, as analyzed by Northern blot 24 h after the H₂O₂ treatment (FIG. 2D). BAPTA-AM also prevented H₂O₂-induced reduction in the genomic HCV RNA level in these cells (FIG. 2E). Therefore, the inhibition of [Ca²⁺]ᵢ elevation resulted in the loss of both rapid and sustained suppression of HCV RNA replication by H₂O₂.

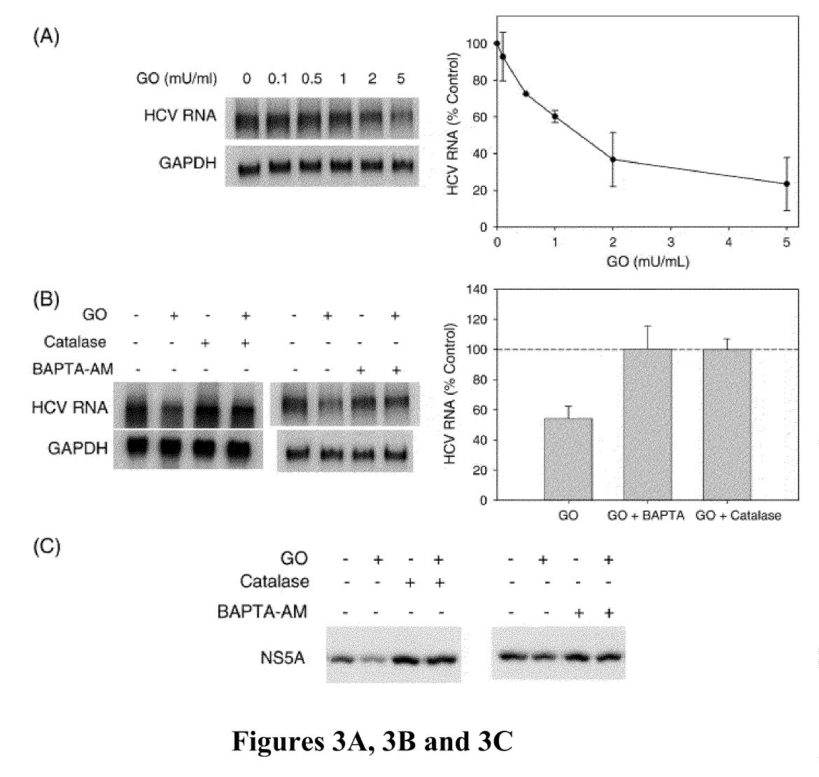

To continue to study the effects of sustained oxidative stress [10], [11], [12] and [13] and the continuous generation of H₂O₂ by NAD(P)H oxidase of Kupffer cells and polymorphonuclear cells [39], [40] and [41] during chronic hepatitis C, GO plus glucose, which continuously generates H₂O₂ enzymatically [33], was used. SgPC2 cells were exposed to different concentrations of GO and analyzed for changes in the HCV RNA level. As shown in FIG. 3A, GO dose-dependently suppressed HCV replication. Exogenous catalase attenuated the suppression by GO, confirming that the suppression was induced by the extracellularly generated H₂O₂ (FIG. 3B). BAPTA-AM was also able to alleviate the suppressive effect of GO on HCV RNA (FIG. 3B). HCV NS5A protein showed changes with GO, BAPTA, and catalase that were similar to those of the HCV RNA (FIG. 3C). Intracellular calcium concentrations, after 24 h of GO treatment, were 66.4±10.7 and 1 19.0±13.6 nM with 2 and 5 mU/ml GO, respectively, which were above that of the control (34.5±2.1 nM; p<0.05). Therefore, both bolus and continuous H₂O₂ treatment suppressed HCV replication through elevated [Ca²⁺]ᵢ.

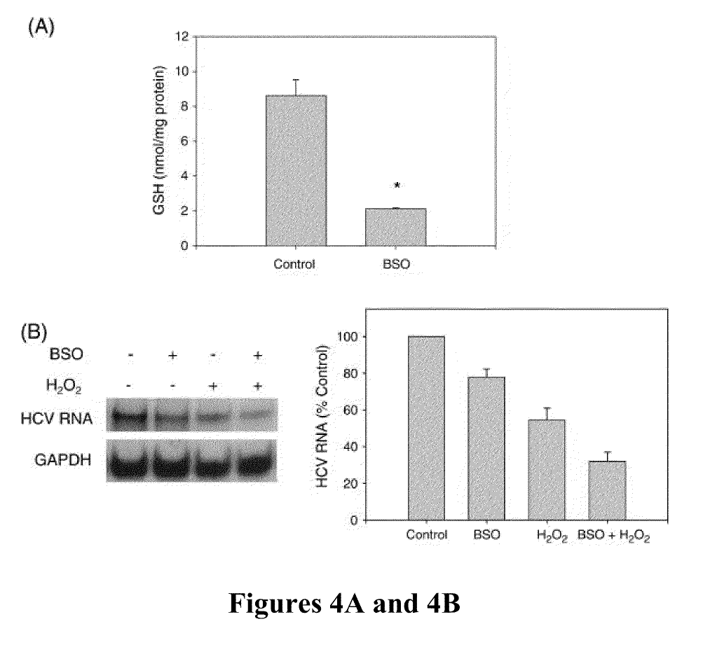

Next, we continued to examine whether endogenous antioxidants could then enhance the activity of HCV RC by antagonizing or reversing the suppression by oxidants. Decreasing total intracellular GSH concentration by about 75% (FIG. 3A) with 1-buthionine S,R-sulfoximine (BSO), an irreversible inhibitor of GSH biosynthesis [42], was sufficient to decrease the HCV RNA level and enhanced the lowering of HCV RNA by H₂O₂ (FIG. 4B). Moreover, this suppression could be overcome with BAPTA-AM (data not shown). Intracellular calcium concentration in BSO-treated cells was 60.6±4.3 nM after 24 h, which was significantly above that of the control (34.5±2.1 nM; p<0.05). Therefore, GSH may enhance HCV RNA replication and decreasing GSH promote the suppression of HCV RNA replication by oxidants.

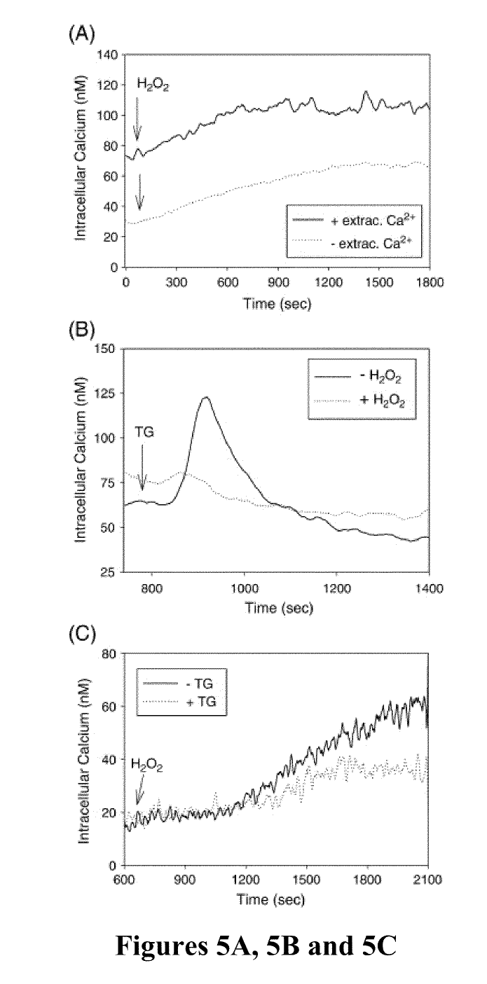

In order to understand the molecular events leading to calcium elevation and subsequent suppression of HCV RNA replication, we next determined the source of the elevated calcium in response to H₂O₂. H₂O₂ caused similar elevation of [Ca²⁺]ᵢ in the presence and absence of extracellular calcium (FIG. 5A). Therefore, H₂O₂ was not likely to elevate [Ca²⁺]ᵢ by causing an influx of extracellular calcium but, rather, by releasing calcium from an internal calcium store(s). Endoplasmic reticulum (ER) is one of the major intracellular calcium stores and plays an important role in calcium signaling. To examine whether the peroxide released calcium from the ER, SgPC2 cells were first exposed to H₂O₂ and then treated with thapsigargin (TG). TG passively depletes the calcium pool in the ER by inhibiting Ca²⁺/ATPase that normally replenishes this calcium pool [25] and [43]. As increases in the [Ca²⁺]ᵢ can trigger the influx of extracellular calcium [25], experiments were conducted in the absence of extracellular calcium to focus on the intracellular events. As expected, TG caused a transient rise in [Ca²⁺]ᵢ (FIG. 5B). Preincubating cells with H₂O₂ significantly decreased the magnitude of the calcium response to TG (FIG. 5B). Similarly, pretreating cells with TG diminished the H₂O₂-induced rises in [Ca²⁺]ᵢ (FIG. 5C). Thus, H₂O₂ increased [Ca²⁺]ᵢ at least partially by triggering the release of calcium from the ER, as previously reported with other cell types by others [26] and [27]. Possible effects of H₂O₂ on other intracellular calcium stores or the Ca²⁺/ATPases in the plasma membrane, however, have not been ruled out. For example, H₂O₂ might inhibit the calcium extrusion by Ca²⁺/ATPases in the plasma membrane and/or the mitochondrial calcium uptake to prolong the cytosolic calcium elevation, after the ER calcium release. Note that the TG-induced rises in the [Ca²⁺]ᵢ were not different in SgPC2 versus naïve Huh7 cells under our experimental conditions.

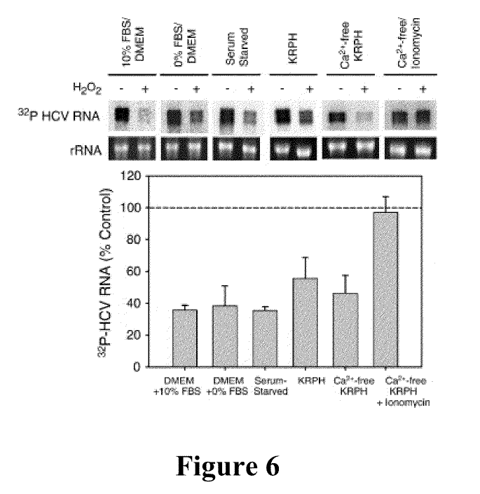

Consistent with the data in FIG. 5, neither the serum depletion nor the absence of extracellular calcium had significant effects on the suppression of HCV replication by H₂O₂ (FIG. 6). When both intracellular and extracellular calcium were depleted by preincubating cells with ionomycin, a calcium ionophore, in a calcium-free buffer, however, the suppressive response to H₂O₂ was significantly alleviated (FIG. 6). These data further confirmed that the inhibition of HCV replication by the peroxide required calcium release from an internal store.

As previously discussed, calcium release from the ER can lead to mitochondrial calcium uptake, leading to mitochondrial dysfunction and apoptosis. To examine whether H₂O₂ triggered mitochondrial dysfunction to result in the rapid inhibition of HCV replication, we first measured the cellular ATP content. H₂O₂ did not cause any decline in the total ATP content after 30 min or 24 h of treatment (125.4±0.3 and 97.3±8.25% of untreated control at 30 min and 24 h, respectively; p>0.05). H₂O₂ also had no significant effect on the mitochondrial membrane potential or apoptosis, either, as measured with JC-1 and annexin V-FITC staining (data not shown). Furthermore, cyclosporin A and ruthenium red, which can inhibit mitochondrial calcium uptake, had no effect on the basal calcium or the magnitude/kinetics of the peroxide-induced calcium elevation (data not shown). These findings are consistent with our previous observation that the concentrations of H₂O₂ we employed did not cause any decrease in cell viability, reduced GSH concentration, or host rRNA synthesis in these cells [8]. Therefore, even though HCV infection has been associated with various mitochondrial abnormalities [19], [44] and [45], H₂O₂ did not seem to inhibit HCV replication by inducing mitochondrial permeability transition in the hepatocytes at the concentrations used in our study.

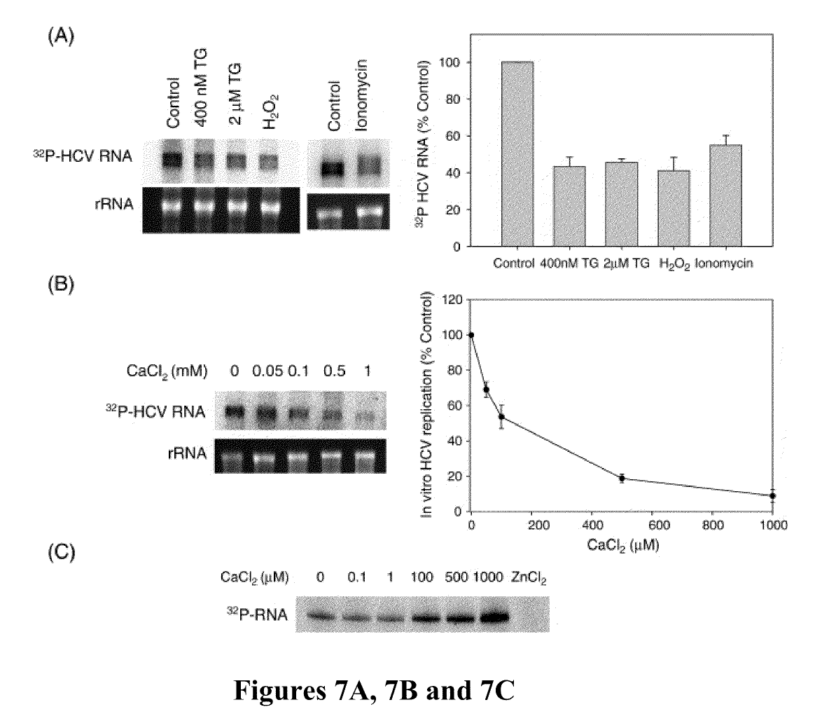

These experiments so far indicated that the oxidative suppression of HCV replication was almost entirely calcium dependent. To determine whether the elevation of [Ca²⁺]ᵢ was not only necessary but also sufficient to cause such suppression, we examined whether other ways of increasing [Ca²⁺]ᵢ had similar inhibitory effects on HCV. As H₂O₂ released calcium from the ER (FIG. 5), SgPC2 cells were first treated with TG for 15 min and analyzed for changes in the in vitro replication potential of HCV. As shown in FIG. 7A, TG decreased the in vitro HCV RNA replication. Ionomycin elicited similar suppressive effects on HCV replication (FIG. 7A). Note that these experiments were focused on early events (i.e., within 15 to 30 min of treatment), as these agents can be toxic and their long-term effects would be difficult to interpret. In addition, adding CaCl₂ directly to the cytoplasmic lysates, before in vitro replication assays, could decrease HCV RNA replication (FIG. 7B). Therefore, increasing [Ca²⁺]ᵢ might be not only necessary for the oxidative suppression of HCV RC but also sufficient to inhibit HCV RC. Consistent with this conclusion, BAPTA-AM tended to increase the basal HCV RNA level, particularly with a prolonged incubation (e.g., 24 h, FIGS. 2D and E). This might be explained by fluctuations in [Ca²⁺]ᵢ and/or ROS generated during cell metabolism and those induced by HCV [17], [23] and [24].

Previously, several divalent cations (Fe²⁺ and Zn²⁺) had been shown to inhibit HCV RNA-dependent RNA polymerase (NS5B) activity [46], [47], [48] and [49]. However, calcium only marginally suppressed the in vitro de novo RNA synthesis by NS5B at 0.1 and 1 μM concentrations (FIG. 7C) and enhanced the polymerase activity at higher concentrations. Therefore, NS5B did not seem to be the calcium-binding target molecule that rendered HCV RC redox- and calcium-sensitive, at least under these conditions.

### Discussion

In this experiment it is shown that H₂O₂ suppresses HCV RC through calcium elevation. Previously, oxidants have been reported to elevate [Ca²⁺]ᵢ by multiple mechanisms, including an influx of extracellular calcium [27], release of calcium bound to annexin VI [28], and release of calcium from the ER [50] (for review, see Ref. [25]). In this study, extracellular calcium had no significant effect on the peroxide-induced calcium elevation. TG decreased peroxide-induced calcium elevation and vice versa, indicating that the peroxide-induced calcium elevation derived, at least in part, from the ER. It is interesting that this release of calcium from the ER did not trigger capacitative calcium entry in these cells. We also found that H₂O₂ did not increase the inositol 1,3-triphosphate (IP₃) level in our study (unpublished observation). The mechanism by which H₂O₂ releases the calcium pool in the ER may thus involve increased sensitivity of IP₃ receptors to IP₃ and oxidation of sulfhydryl moieties on IP₃ receptors, as proposed by others [50] and [51]. The peroxide-induced calcium elevation, however, did not follow a transient pattern, which would be expected from an ER-mediated calcium release, and the present data do not preclude the possible effects of H₂O₂ on other components of calcium metabolism, such as the inhibition of Ca²⁺/ATPases in the plasma membrane and inhibition of calcium uptake by the mitochondria. It should be noted that previously, nonstructural proteins of HCV have been suggested to induce ER stress, with a subsequent release of calcium from the ER that would increase ROS generation by mitochondria [52]. In this study, however, we did not detect any significant change in the basal [Ca²⁺]ᵢ or the magnitude of thapsigargin-induced calcium elevation in replicon versus naïve Huh7 cells, suggesting that the calcium pool in the ER remained largely unaffected by the HCV nonstructural proteins. In addition, our data suggest that mitochondrial permeability transition did not occur with the peroxide treatment.

How calcium affects the activity of HCV RC remains unknown at the time of the completion of this experiment. The decrease in NS5A protein level (FIG. 3C) after 24 h of GO exposure was most likely a result of decreased HCV replication than the cause, as the protein level did not change at 30 min of H₂O₂ treatment [8]. In our previous study, the oxidative suppression of HCV replication was not associated with a change in the stability of HCV RNA transcript or decreased amounts of HCV proteins and the RNA template. Instead, the suppression was associated with the disappearance of HCV replicating activity that cofractionated with the Golgi membranes [8]. Our findings are consistent with findings of Serafino et al. and Aizaki et al. [53] and [54]. Lipid rafts and membranous webs have also been implicated as important sites of HCV RNA replication [54], [55] and [56]. It is possible that calcium binds to host and/or viral proteins to cause a disruption of HCV RC in the membranes. Experiments are under way to identify the calcium effector protein(s) that mediates the suppression of HCV replication by H₂O₂. Interestingly, although Ca²⁺ dose-dependently suppressed HCV replication when directly added to in vitro reactions (FIG. 7B), whether the magnitude of calcium elevation always correlates with the level of suppression of HCV replication in vitro and in vivo (FIG. 2 and FIG. 7) is not clear. Similar disparity between in vivo and in vitro concentrations of calcium required for various protein functions has been observed with well-characterized calcium-binding proteins like calpain and calpastatin [57] and might be explained by highly localized calcium transients inside the cells, perturbation of normal cell metabolism and signaling processes in the in vitro studies, relatively high affinity of the calcium effector protein(s) to calcium (e.g., compared to Indo-1 AM, which binds only to free calcium), and the existence of multiple calcium effector proteins with different calcium-binding activity. Surprisingly, unlike Fe²⁺ and Zn²⁺, which have been shown to suppress NS5B polymerase activity, we found that calcium did not significantly suppress the polymerase activity, at least in vitro (FIG. 7C). The enhancement we saw with 100 μM calcium concentration is intriguing but its biological relevance is questionable. Preliminary studies using multiple inhibitor compounds suggested that at least two of the well-characterized calcium-binding proteins, calpain and calmodulin, are not involved. During this investigation, Waris et al. reported a suppression of HCV RNA replication by NF-κB-mediated induction of cyclooxygenase-2 gene expression [58]. However, this mechanism cannot explain the rapid suppression of HCV RC we observed within 15-30 min. Thus, whereas finding the ultimate target molecule(s) might reveal potential targets for antiviral therapy, the identity of the calcium-binding target molecule remains elusive. Importantly, TG caused a more transient elevation in [Ca²⁺]ᵢ than H₂O₂, which was followed by a decline; likewise, TG elicited a transient suppression of HCV replication, followed by a rebound, with prolonged incubation (data not shown). Therefore, while looking for potential antiviral targets, it will be important to consider the kinetics of [Ca²⁺]ᵢ modulation as well as the kinetics of the observed antiviral response.

In the previous study, NAC, a precursor of cysteine for GSH biosynthesis and a thiol reductant, was able to partially antagonize the oxidative suppression of HCV RNA replication [8]. In the present study, we also showed that decreasing intracellular concentration of GSH, the most abundant intracellular thiol reductant, was sufficient to suppress HCV RNA level and to enhance the suppression of HCV RC by H₂O₂ (FIG. 4). The data suggest that endogenous generation of oxidants, either due to normal cell metabolism or triggered by HCV protein expression [15], [16], [18], [19], [20] and [21], might be sufficient to modulate HCV RC in these cells. Most of all, however, the data indicate that ROS might suppress HCV RC, whereas antioxidants favor HCV RNA replication by antagonizing the suppressive effects of oxidants. This is the first study to demonstrate a modulation of viral replication machinery, independent of transcription factor modulation or oxidative damage to the virus particles, through redox signaling.

Nevertheless, the subgenomic and genomic replicons employed in this study do not generate virus particles and, therefore, although ROS clearly suppressed the activity of HCV RC in this study, the effects of ROS on the infectivity and morphogenesis of HCV remain to be determined. In this regard, it should be noted that some studies have correlated antioxidant therapy with a decreased viral titer, suggesting potential proviral effects of oxidants [59] and [60]. Although these findings are difficult to reconcile, some of the discrepancies may be explained by the effects that some HCV proteins have on the host redox status [15], [16], [17], [18], [19], [20] and [21]. It is also possible that antioxidants have other effects, such as on the immune system, which lead to an overall reduction of HCV viral load in vivo. Furthermore, oxidants/antioxidants may have other effects on different steps of HCV life cycle. In fact, whether calcium, the concentration of which changes physiologically with signaling, binds to cellular proteins or various constituents of HCV RC to differentially regulate the RNA replication versus other steps of viral life cycle remains to be examined. Further studies that characterize the effects of redox and calcium signaling on other steps of HCV life cycle will help resolve the complex mode of virus-host interactions involving ROS and calcium signaling during acute and chronic hepatitis C.

In conclusion, both endogenous and exogenous H₂O₂ can modulate HCV RC through redox signaling. Our findings indicate the importance of calcium and redox homeostasis during HCV infection and support a novel function of ROS, generated during inflammation, in the modulation of the viral replication complex through signaling. Currently, 170 million people are estimated to be infected with HCV, including 4 million in the United States.

### Experiment 2

Introduction:

HCV is a positive-sense stranded RNA virus of Flaviviridae family. The HCV genome is about 9.6 kb in length and consists of the 5′ untranslated region (UTR), the structural (C, E1, E2) and nonstructural (p7, NS2, NS3, NS4A/B, NS5A/B) protein-coding regions, and the 3′ UTR. The HCV virion is taken into the cell through clathrin-mediated endocytosis and is uncoated, releasing the genome into the cytosol. The viral genome is then translated in a cap-independent manner via an internal ribosomal entry site (IRES) located in the 5′ UTR of HCV. The resulting viral polyprotein is cleaved into its functional viral proteins by host and viral proteases. Occasionally, the ribosome slips into alternate reading frames producing different proteins, though their functions are unknown (61-64). HCV RNA replication is mediated by NS5B, an RNA-dependent RNA polymerase, and other proteins that comprise the replication complex, located in the cytoplasm on a virus-induced membrane structure called the membranous web (65). The capsid, which is formed by the core protein (C), internalized the viral genome and most likely then buds into the endoplasmic reticulum (ER) or an ER-derived compartment. Then, the enveloped virions are exported from the cell via the normal host secretory pathway (66).

Previously, it was found that HCV infection, like many other viral infections, is associated with severe alterations of the host redox status with increased generation of reactive oxygen/nitrogen species (ROS/RNS) and decreased host antioxidant defense (67). In addition, ROS was shown to inhibit the RNA replication of genotype 1b HCV in hepatocytes (68, 69). These observations were made, using subgenomic and genomic HCV replicons that can replicate in Huh7 human hepatoma cells (68, 70, 71). These replicon systems, however, do not produce virus particles, and whether ROS suppress HCV replication in the context of the complete HCV lifecycle remained unknown. Plus, HCV is remarkably heterogeneous in sequences and have been classified into different genotypes (72). Thus, whether different HCV genotypes respond to ROS similarly or differently as genotype 1b HCV sequences was not known.

Therefore, in this study, the interactions between HCV and ROS was examined, using recently described virus-producing cell culture systems of HCV. JFH1 strain is developed from a strain of genotype 2a HCV from a patient with fulminant hepatitis (73). Other systems that also produce infectious virus particles have also been described (74, 75). Using the virus-producing in vitro cell culture system, hydrogen peroxide is shown to decrease HCV RNA level in the context of the complete lifecycle of HCV. Hydrogen peroxide, generated with bolus H₂O₂ or glucose oxidase plus glucose, also decreased HCV RNA in the subgenomic JFH1-Luc (genotype 2a) and Con1-Neo (genotype 1b) RNA-transfected cells, suggesting that the HCV RNA genome replication is affected, independent of the genotype. Decreasing intracellular glutathione (GSH), a major endogenous antioxidant defense, with L-buthionine S,R-sulfoximine (BSO) likewise decreased HCV RNA's by decreasing GSH, suggesting that endogenous oxidative stress suppresses HCV replication. Furthermore, BSO enhanced the suppression of HCV replication by H₂O₂. Therefore, oxidative stress suppresses HCV RNA replication of genotypes 1 and 2 at the level of RNA genome replication.

### Materials and Methods

Reagents and HCV constructs: JFH1 and its replication defective derivative (JFH1/GND) constructs were obtained from Dr. Takaji Wakita (Tokyo Metropolitan Institute of Neurosciences, Japan) (73). pSGR-JFH1-Luc (subgenomic JFH1 replicon, also referred to as SgJFH1-Luc) was also obtained from Dr. Wakita. SgPC2 cells, stably transfected with SgCon1-Neo RNA, were also used (68). Cell culture reagents and primers were obtained from Invitrogen. Real time PCR reagents were obtained from Applied Biosystems. NS5A antibodies were purchased from Biodesign, Inc. Other reagents were obtained from Sigma-Aldrich, unless indicated otherwise.

Cell Culture, electroporation of HCV RNA's, and infection—Huh7 human hepatoma cells were cultured in Dulbecco's modified eagle medium (DMEM, Invitrogen, Carlsbad, Calif.), supplemented with 10% fetal bovine serum (FBS, Invitrogen), 100 units/ml of penicillin, and 100 μg/ml of streptomycin in 5% CO₂ incubator. SgPC2 cells were maintained in the above medium, supplemented with 0.5 mg/ml of G418 (Invitrogen). G418 was removed at least one day prior to cell treatments.

For viral RNA transfection, JFH1, JFH1/GND, and pSGR-JFH1-Luc were linearized with XbaI and used to synthesize the corresponding positive-sense stranded HCV RNA, as previously described (68). Then, 5×10⁶ Huh7 cells were rinsed with Opti-Mem (Invitrogen), mixed briefly with 10-20 μg of the in vitro transcribed RNA in 0.4 ml of Opti-Mem, and then electroporated at 260 volts and 950 μF. Then, 1 million cells were seeded onto 60-mm cell culture dishes in DMEM containing 10% FBS. Medium was changed daily. For infection with HCV, medium collected from JFH1 RNA-transfected cells were cleared of cell debris, as described, and added to naïve Huh7 cells. Typically, 1:2 or 3:2 ratio of the virus-containing medium and fresh cell culture medium were used.

For cell treatments, the transfected cells were exposed to H₂O₂ or glucose oxidase plus glucose for 24 hrs, and the amount of the HCV RNA was analyzed by Northern blot or quantitative real time reverse-transcription-polymerase chain reaction (qRT-PCR) (see below). JFH1/GND was used as the negative control. When L-buthionine S,R-sulfoximine (BSO) was used, the cells were pretreated with BSO for 16-24 hrs prior to the initiation of treatments.

HCV RNA quantification by Northern blot and qRT-PCR—Total RNA was extracted from cells, using Trizol reagent (Invitrogen), following the manufacturer's protocol. RNA levels were quantified using Nanodrop (Nanodrop Technologies). Four to 10 μg of RNA were then subjected to Northern-blot analysis using a ³²P-labeled double-stranded DNA probe, prepared from nt. 3669 to 6016 of the Con1 subgenomic replicon or nt. 334 to 2792 of the JFH1 sequence. RNA bands were imaged and quantified with a phosphoimager (Cyclone, Perkin Elmer). Glyceraldehyde 3-phosphodehydrogenase (GAPDH) mRNA levels were used as the loading controls.

Intracellular JFH1 HCV RNA content was also analyzed by qRT-PCR (Taqman), using ABI7300 real time PCR system and one step RT-PCR kit from Applied Biosystems. The forward primer was 5′-TCTGCGGAACCGGTGAGTA-3′ (nt. 146 to 164) and the reverse primer, 5′-TCAGGCAGTACCACAAGGC-3′ (nt. 277 to 295). The probe was 5′CCAGTCTTCCCGGCAATTCCG3′ (nt. 168 to 188) and was fluorogenically labeled with FAM (5′) and TAMRA (3′) (Biosearch Technologies, Novato, Calif.). Briefly, the reactions were carried out at 48° C. for 30 min for reverse transcription, followed by denaturation at 95° C. for 10 min, and then 50 cycles of 15 sec denaturation at 95° C. plus 60 sec of annealing and extension at 60° C. All samples were analyzed in duplicates or triplicates. In vitro RNA transcripts were used to generate standard curve to determine RNA copy numbers. The level of 18S rRNA was also analyzed, using manufacturer-optimized protocol, as the control.

To analyze the amount of the extracellular HCV RNA, medium from the cells were cleared by low-speed centrifugation and then, treated with RNaseA at room temperature. Then, the RNA samples were extracted, using Trizol LS Reagent (Invitrogen), and analyzed for HCV RNA content by qRT-PCR.

### Luciferase Assay:

SgJFH1-Luc RNA replication was also monitored by firefly luciferase reporter assay, using a kit from Promega.

### Immunofluorescence Assay:

JFH1-transfected cells and mock-transfected cells were fixed with formaldehyde and analyzed for HCV core protein expression by immunofluorescence staining, using rabbit anti-core antibodies (obtained from J. Ou, University of Southern California), FITC-conjugated goat anti-rabbit secondary antibodies (Santa Cruz Biotechnology) via confocal microscopy (Nikon).

Determination of ATP concentrations—ATP level was measured, using Somatic Cell ATP Assay kit from Sigma-Aldrich, as previously described (68). Protein level was determined with bicinchoninic acid kit (Pierce Biotechnology, Inc., Rockford, Ill.).

### Statistics:

Data are presented as mean±SEM (standard error of the mean) of several independent experiments. Data were analyzed, using Student's t-test or one-way analysis of variance (ANOVA). P value≦0.05 was considered significant. All experiments were repeated at least 2-3 times.

### Results and Discussion

Replication and Infectivity of JFH1 HCV RNA's in Huh7 Human Hepatoma Cells:

DNA plasmids containing the JFH1, JFH1-GND, and subgenomic JFH1 replicon sequences (73) were digested with XbaI and transcribed in vitro using a T7 polymerase to produce full genome-length JFH1 and JFH1/GND HCV RNA's. Upon transfection of these RNA transcripts into naïve Huh7 hepatoma cells, continued replication of JFH1 HCV RNA could be demonstrated (data not shown). Time-dependent increases in both intracellular and extracellular generation of HCV RNA could be demonstrated. JFH1/GND RNA did not replicate, as expected. The medium from JFH1 RNA-transfected cells was also able to infect naïve Huh7 cells. Expression of HCV core protein could also be readily demonstrated by immunofluorescence staining (data not shown). Continued replication of the subgenomic JFH1 RNA could be demonstrated by luciferase assay (data not shown).

### Hydrogen Peroxide Decreases JFH1 HCV RNA:

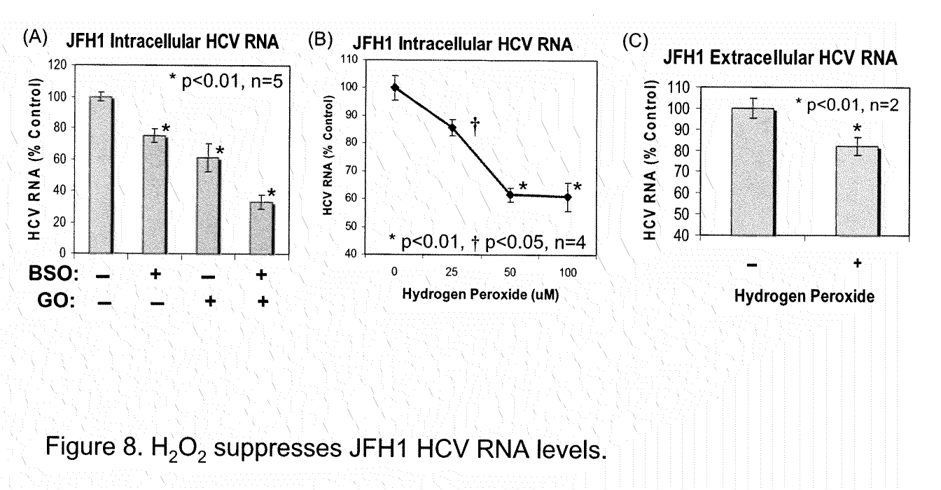

Previous studies showed a dose-dependent inhibition of HCV replication by H₂O₂ in cells stably-transfected with Con1 (genotype 1b) subgenomic replicon RNA (SgPC2 cells) and cells transiently transfected with H77c/Con1 (genotype 1a/b) hybrid genomic RNA (68), and the suppression depended on intracellular calcium elevation (69). To test whether hydrogen peroxide also suppressed HCV replication, JFH1-transfected cells were treated with 25 uM, 50 uM and 100 uM H₂O₂ for 24 hrs. Both intracellular and extracellular HCV RNA levels were significantly decreased with H₂O₂ (FIGS. 8B and C). Low concentrations (e.g., 0.25 mU/mL) of glucose oxidase (GO) plus glucose, which enzymatically produces H₂O₂ extracellularly, also decreased intracellular HCV RNA content by 30±8%. Furthermore, 20 uM of L-buthionine S,R-sulfoximine (BSO) produced in a significant suppression of intracellular HCV RNA level by itself that was exacerbated by GO plus glucose (FIG. 8A). BSO is an inhibitor of GSH biosynthesis and therefore, decreases antioxidant defense of the cells, which would amplify the effects of the endogenously generated ROS. Twenty to 100 μM BSO has been shown to decrease intracellular GSH content by about 60% in Huh7 cells (unpublished observation). These results suggest that exogenous H₂O₂ as well as endogenously generated ROS can suppress HCV replication in the context of the complete viral lifecycle.

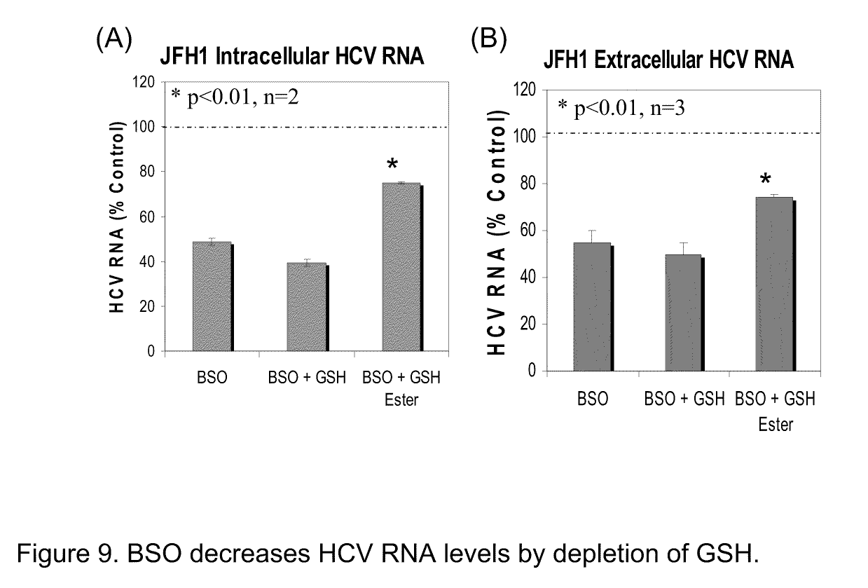

To confirm that BSO was acting specifically by decreasing GSH content, GSH was added back in the form of GSH ester which would then be cleaved by cellular esterases to generate GSH inside the cells. If BSO decreased HCV replication by decreasing GSH, rather than some nonspecific effects, GSH ester would be expected to restore HCV replication in the BSO-treated cells. Indeed, it was found that GSH ester was able to blunt the suppression of intracellular and extracellular HCV RNA levels by BSO (FIG. 9). In contrast, exogenous addition of GSH was not able to restore HCV replication. GSH, unlike GSH ester, is not taken up by cells and typically requires its breakdown by γ-glutamyltranspeptidase and dipeptidase, and the individual components need to be taken up by the cells and used for intracellular de novo synthesis of GSH. De novo synthesis of GSH is inhibited by BSO. Therefore, the data suggest that BSO is decreasing HCV replication by depleting GSH by inhibition of its biosynthesis.

ATP concentration was measured to determine if the treatments were cytotoxic to the cells. The BSO and GO concentrations used did not cause any loss in cell survivability, nor did any of the H₂O₂ treatments with the exception of 100 μM (data not shown). Note that the same concentration of H₂O₂ was previously shown not to cause any depletion of ATP content in the subgenomic replicon cells (68), which suggests that the JFH1-transfected cells are more susceptible to oxidative stress.

### Hydrogen Peroxide Suppresses HCV Replication at the Level of RNA Genome Replication:

To investigate whether the HCV RNA replication is modulated by ROS, Huh7 cells were transfected with the SgJFH1-Luc RNA and treated with GO plus glucose. SgJFH1-Luc RNA does not generate infectious virus particles and only supports the genome replication of HCV.

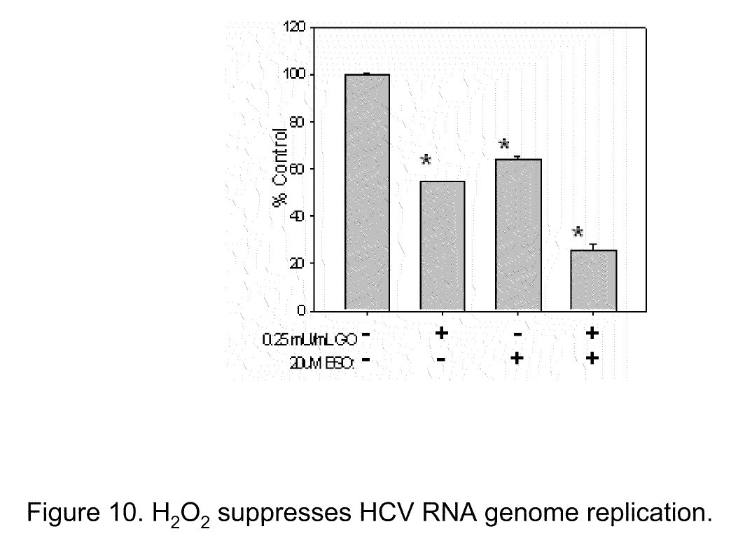

After 24 and 48 hours, respectively, the cells were collected in PBS, centrifuged, lysed in the reporter lysis buffer (Promega), and analyzed for luciferase activity. GO plus glucose decreased HCV RNA level, suggesting that H₂O₂ suppresses HCV replication at the level of RNA genome replication, as previously shown with Con1 sequence of genotype 1b (FIG. 10) (69). Once again, BSO further suppressed HCV RNA. Furthermore, the data suggest that H₂O₂ suppresses the replication of both genotypes 1 and 2.

### Diphenylene Iodonium (DPI) Enhances Con1 Subgenomic RNA Replication in Huh7 Cells:

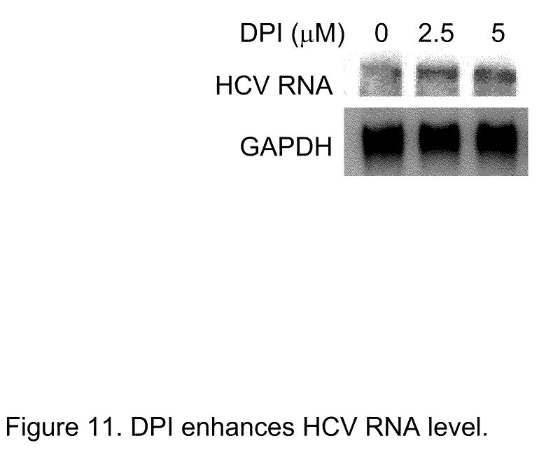

Studies have suggested that various flavoproteins (e.g., nitric oxide synthase, cytochrome P450, and NAD(P)H oxidase 2) may act as the source of ROS/RNS during HCV infection (76). To test whether suppressing ROS generation by such flavoproteins would then facilitate HCV replication, Huh7 cells that are stably transfected with HCV subgenomic RNA replicon of Con1 sequence (SgPC2 cells) were treated with different concentrations of DPI, an inhibitor of Nox and other flavoproteins, and examined for HCV replication. DPI significantly elevated HCV RNA content in these cells (FIG. 11), again suggesting that the endogenous generation of ROS/RNS suppress HCV replication.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

[1] B. Halliwell and J. M. C. Gutteridge, Free Radicals in Biology and Medicine, Clarendon Press, Oxford/New York (1999).
[2] B. M. Babior, NADPH oxidase: an update, Blood 93 (1999), pp. 1464-1476.
[3] H. J. Forman and E. Cadenas, Oxidative Stress and Signal Transduction, Chapman & Hall, New York (1997).
[4] M. Sundaresan, Z. X. Yu, V. J. Ferrans, K. Irani and T. Finkel, Requirement for generation of H2O2 for platelet-derived growth factor signal transduction, Science 270 (1995), pp. 296-299.
[5] S. G. Rhee, S. W. Kang, W. Jeong, T. S. Chang, K. S. Yang and H. A. Woo, Intracellular messenger function of hydrogen peroxide and its regulation by peroxiredoxins, Curr. Opin. Cell Biol. 17 (2005), pp. 183-189.
[6] Y. W. Zheng and T. S. Yen, Negative regulation of hepatitis B virus gene expression and replication by oxidative stress, J. Biol. Chem. 269 (1994), pp. 8857-8862.
[7] R. Schreck, P. Rieber and P. A. Baeuerle, Reactive oxygen intermediates as apparently widely used messengers in the activation of the NF-kappa B transcription factor and HIV-1, EMBO J. 10 (1991), pp. 2247-2258.
[8] J. Choi, K. J. Lee, Y. Zheng, A. K. Yamaga, M. M. C. Lai and J.-H. Ou, Reactive oxygen species suppress hepatitis C virus RNA replication in human hepatoma cells, Hepatology 39 (2004), pp. 81-89.
[9] B. Robertson, G. Myers, C. Howard, T. Brettin, J. Bukh, B. Gaschen, T. Gojobori, G. Maertens, M. Mizokami, O. Nainan, S. Netesov, K. Nishioka, T. Shin i, P. Simmonds, D. Smith, L. Stuyver and A. Weiner, Classification, nomenclature, and database development for hepatitis C virus (HCV) and related viruses: proposals for standardization. International Committee on Virus Taxonomy, Arch. Virol. 143 (1998), pp. 2493-2503.
[10] F. Farinati, R. Cardin, N. De Maria, G. Della Libera, C. Marafin, E. Lecis, P. Burra, A. Floreani, A. Cecchetto and R. Naccarato, Iron storage, lipid peroxidation and glutathione turnover in chronic anti-HCV positive hepatitis, J. Hepatol. 22 (1995), pp. 449-456.
[11] F. Farinati, R. Cardin, P. Degan, N. De Maria, R. A. Floyd, D. H. Van Thiel and R. Naccarato, Oxidative DNA damage in circulating leukocytes occurs as an early event in chronic HCV infection, Free Radical Biol. Med. 27 (1999), pp. 1284-1291.
[12] K. Swietek and J. Juszczyk, Reduced glutathione concentration in erythrocytes of patients with acute and chronic viral hepatitis, J. Viral Hepat. 4 (1997), pp. 139-141.
[13] G. Barbaro, G. Di Lorenzo, M. Ribersani, M. Soldini, G. Giancaspro, G. Bellomo, G. Belloni, B. Grisorio and G. Barbarini, Serum ferritin and hepatic glutathione concentrations in chronic hepatitis C patients related to the hepatitis C virus genotype, J. Hepatol. 30 (1999), pp. 774-782.
[14] J. Emerit, C. Beaumont and F. Trivin, Iron metabolism, free radicals, and oxidative injury, Biomed. Pharmacother. 55 (2001), pp. 333-339.
[15] I. Qadri, M. Iwahashi, J. M. Capasso, M. W. Hopken, S. Flores, J. Schaack and F. R. Simon, Induced oxidative stress and activated expression of manganese superoxide dismutase during hepatitis C virus replication: role of JNK, p38 MAPK and AP-1, Biochem. J. 378 (2004) (Pt 3), pp. 919-928.
[16] C. Bureau, J. Bernad, N. Chaouche, C. Orfila, M. Beraud, C. Gonindard, L. Alric, J. P. Vinel and B. Pipy, Nonstructural 3 protein of hepatitis C virus triggers an oxidative burst in human monocytes via activation of NADPH oxidase, J. Biol. Chem. 276 (2001), pp. 23077-23083.
[17] G. Gong, G. Waris, R. Tanveer and A. Siddiqui, Human hepatitis C virus NS5A protein alters intracellular calcium levels, induces oxidative stress, and activates STAT-3 and NF-kappa B, Proc. Natl. Acad. Sci. U.S.A. 98 (2001), pp. 9599-9604.
[18] M. Okuda, K. Li, M. R. Beard, L. A. Showalter, F. Scholle, S. M. Lemon and S. A. Weinman, Mitochondrial injury, oxidative stress, and antioxidant gene expression are induced by hepatitis C virus core protein, Gastroenterology 122 (2002), pp. 366-375.
[19] K. Moriya, K. Nakagawa, T. Santa, Y. Shintani, H. Fujie, H. Miyoshi, T. Tsutsumi, T. Miyazawa, K. Ishibashi, T. Horie, K. Imai, T. Todoroki, S. Kimura and K. Koike, Oxidative stress in the absence of inflammation in a mouse model for hepatitis C virus-associated hepatocarcinogenesis, Cancer Res. 61 (2001), pp. 4365-4370.
[20] F. Wen, M. Y. Abdalla, C. Aloman, J. Xiang, I. M. Ahmad, J. Walewski, M. L. McCormick, K. E. Brown, A. D. Branch, D. R. Spitz, B. E. Britigan and W. N. Schmidt, Increased prooxidant production and enhanced susceptibility to glutathione depletion in HepG2 cells co-expressing HCV core protein and CYP2E1, J. Med. Virol. 72 (2004), pp. 230-240.
[21] K. Li, T. Prow, S. M. Lemon and M. R. Beard, Cellular response to conditional expression of hepatitis C virus core protein in Huh7 cultured human hepatoma cells, Hepatology 35 (2002), pp. 1237-1246.
[22] K. Machida, K. T. Cheng, V. M. Sung, K. J. Lee, A. M. Levine and M. M. Lai, Hepatitis C virus infection activates the immunologic (type II) isoform of nitric oxide synthase and thereby enhances DNA damage and mutations of cellular genes, J. Virol. 78 (2004), pp. 8835-8843.
[23] A. Bergqvist, S. Sundstrom, L. Y. Dimberg, E. Gylfe and M. G. Masucci, The hepatitis C virus core protein modulates T cell responses by inducing spontaneous and altering T-cell receptor-triggered Ca2+ oscillations, J. Biol. Chem. 278 (2003), pp. 18877-18883.
[24] S. D. Griffin, L. P. Beales, D. S. Clarke, O. Worsfold, S. D. Evans, J. Jaeger, M. P. Harris and D. J. Rowlands, The p7 protein of hepatitis C virus forms an ion channel that is blocked by the antiviral drug, Amantadine, FEBS Lett. 535 (2003) (1-3), pp. 34-38.
[25] J. Giron-Calle and H. J. Forman, Cell Ca2+ in signal transduction: modulation in oxidative stress. In: C. K. Sen, H. Sies and P. A. Baeuerle, Editors, Antioxidant and Redox Regulation of Genes, Academic Press, San Diego (2000), pp. 106-127.
[26] D. E. Wesson and S. J. Elliott, The H2O2-generating enzyme, xanthine oxidase, decreases luminal Ca2+ content of the IP3-sensitive Ca2+ store in vascular endothelial cells, Microcirculation 2 (1995), pp. 195-203.
[27] R. Carini, G. Bellomo, L. Paradisi, M. U. Dianzani and E. Albano, 4-Hydroxynonenal triggers Ca2+ influx in isolated rat hepatocytes, Biochem. Biophys. Res. Commun. 218 (1996), pp. 772-776.
[28] C. R. Hoyal, A. P. Thomas and H. J. Forman, Hydroperoxide-induced increases in intracellular calcium due to annexin VI translocation and inactivation of plasma membrane Ca2+-ATPase, J. Biol. Chem. 271 (1996), pp. 29205-29210.
[29] V. Lohmann, F. Korner, J. Koch, U. Herian, L. Theilmann and R. Bartenschlager, Replication of subgenomic hepatitis C virus RNAs in a hepatoma cell line, Science 285 (1999), pp. 110-113.
[30] K. J. Blight, A. A. Kolykhalov and C. M. Rice, Efficient initiation of HCV RNA replication in cell culture, Science 290 (2000), pp. 1972-1974.
[31] M. Yanagi, R. H. Purcell, S. U. Emerson and J. Bukh, Transcripts from a single full-length cDNA clone of hepatitis C virus are infectious when directly transfected into the liver of a chimpanzee, Proc. Natl. Acad. Sci. U.S.A. 94 (1997), pp. 8738-8743.
[32] J. Choi, R. M. Liu and H. J. Forman, Adaptation to oxidative stress: quinone-mediated protection of signaling in rat lung epithelial L2 cells, Biochem. Pharmacol. 53 (1997), pp. 987-993.
[33] H. Hiraishi, A. Terano, M. Razandi, T. Sugimoto, T. Harada and K. J. Ivey, Role of cellular superoxide dismutase against reactive oxygen metabolite injury in cultured bovine aortic endothelial cells, J. Biol. Chem. 267 (1992), pp. 14812-14817.
[34] N. Ali, K. D. Tardif and A. Siddiqui, Cell-free replication of the hepatitis C virus subgenomic replicon, J. Virol. 76 (2002), pp. 12001-12007.
[35] F. Tietze, Enzymatic method for quantitative determination of nanogram amounts of total and oxidized glutathione: application to mammalian blood and other tissues, Anal Biochem. 27 (1967), pp. 502-522.
[36] N. V. Vo, J. R. Tuler and M. M. Lai, Enzymatic characterization of the full-length and C-terminally truncated hepatitis C virus RNA polymerases: function of the last 21 amino acids of the C terminus in template binding and RNA synthesis, Biochemistry 43 (2004), pp. 10579-10591.
[37] M. Frese, V. Schwarzle, K. Barth, N. Krieger, V. Lohmann, S. Mihm, O. Haller and R. Bartenschlager, Interferon-gamma inhibits replication of subgenomic and genomic hepatitis C virus RNAs, Hepatology 35 (2002), pp. 694-703.
[38] H. Farghali, E. Kmonickova, H. Lotkova and J. Martinek, Evaluation of calcium channel blockers as potential hepatoprotective agents in oxidative stress injury of perfused hepatocytes, Physiol. Res. 49 (2000), pp. 261-268.
[39] J. M. Roberts, J. W. Searle and W. G. Cooksley, Histological patterns of prolonged hepatitis C infection, Gastroenterol. Jpn. 28 (1993) (Suppl. 5), pp. 37-41.
[40] W. F. Reynolds, K. Patel, S. Pianko, L. M. Blatt, J. J. Nicholas and J. G. McHutchison, A genotypic association implicates myeloperoxidase in the progression of hepatic fibrosis in chronic hepatitis C virus infection, Genes Immun. 3 (2002), pp. 345-349.
[41] F. Toro, A. Conesa, A. Garcia, N. E. Bianco and J. B. De Sanctis, Increased peroxide production by polymorphonuclear cells of chronic hepatitis C virus-infected patients, Clin. Immunol. Immunopathol. 88 (1998), pp. 169-175.
[42] O. W. Griffith and A. Meister, Potent and specific inhibition of glutathione synthesis by buthionine sulfoximine (S-n-butyl homocysteine sulfoximine), J. Biol. Chem. 254 (1979), pp. 7558-7560.
[43] G. Inesi and Y. Sagara, Thapsigargin, a high affinity and global inhibitor of intracellular Ca²⁺ transport ATPases, Arch. Biochem. Biophys. 298 (1992), pp. 313-317.
[44] M. Korenaga, T. Wang, Y. Li, L. A. Showalter, T. Chan, J. Sun and S. A. Weinman, Hepatitis C virus core protein inhibits mitochondrial electron transport and increases ROS production, J. Biol. Chem. 280 (2005), pp. 37481-37488.
[45] T. Naas, M. Ghorbani, I. Alvarez-Maya, M. Lapner, R. Kothary, Y. De Repentigny, S. Gomes, L. Babiuk, A. Giulivi, C. Soare, A. Azizi and F. Diaz-Mitoma, Characterization of liver histopathology in a transgenic mouse model expressing genotype 1a hepatitis C virus core and envelope proteins 1 and 2, J. Gen. Virol. 86 (2005) (Pt 8), pp. 2185-2196.
[46] C. Fillebeen, A. M. Rivas-Estilla, M. Bisaillon, P. Ponka, M. Muckenthaler, M. W. Hentze, A. E. Koromilas and K. Pantopoulos, Iron inactivates the RNA polymerase NS5B and suppresses subgenomic replication of hepatitis C virus, J. Biol. Chem. 280 (2005), pp. 9049-9057.
[47] E. Ferrari, J. Wright-Minogue, J. W. Fang, B. M. Baroudy, J. Y. Lau and Z. Hong, Characterization of soluble hepatitis C virus RNA-dependent RNA polymerase expressed in Escherichia coli, J. Virol. 73 (1999), pp. 1649-1654.
[48] R. W. Hardy, J. Marcotrigiano, K. J. Blight, J. E. Majors and C. M. Rice, Hepatitis C virus RNA synthesis in a cell-free system isolated from replicon-containing hepatoma cells, J. Virol. 77 (2003), pp. 2029-2037.
[49] V. C. Lai, S. Dempsey, J. Y. Lau, Z. Hong and W. Zhong, In vitro RNA replication directed by replicase complexes isolated from the subgenomic replicon cells of hepatitis C virus, J. Virol. 77 (2003), pp. 2295-2300.
[50] D. C. Renard-Rooney, S. K. Joseph, M. B. Seitz and A. P. Thomas, Effect of oxidized glutathione and temperature on inositol 1,4,5-trisphosphate binding in permeabilized hepatocytes, Biochem. J. 310 (1995) (Pt 1), pp. 185-192.
[51] P. C. Redondo, G. M. Salido, J. A. Rosado and J. A. Pariente, Effect of hydrogen peroxide on Ca²⁺ mobilization in human platelets through sulfhydryl oxidation dependent and independent mechanisms, Biochem. Pharmacol. 67 (2004), pp. 491-502.
[52] K. D. Tardif, G. Waris and A. Siddiqui, Hepatitis C virus, ER stress, and oxidative stress, Trends Microbiol. 13 (2005), pp. 159-163.
[53] A. Serafino, M. B. Valli, F. Andreola, A. Crema, G. Ravagnan, L. Bertolini and G. Carloni, Suggested role of the Golgi apparatus and endoplasmic reticulum for crucial sites of hepatitis C virus replication in human lymphoblastoid cells infected in vitro, J. Med. Virol. 70 (2003), pp. 31-41.
[54] H. Aizaki, K. J. Lee, V. M. Sung, H. Ishiko and M. M. Lai, Characterization of the hepatitis C virus RNA replication complex associated with lipid rafts, Virology 324 (2004), pp. 450-461.
[55] R. Gosert, D. Egger, V. Lohmann, R. Bartenschlager, H. E. Blum, K. Bienz and D. Moradpour, Identification of the hepatitis C virus RNA replication complex in Huh-7 cells harboring subgenomic replicons, J. Virol. 77 (2003), pp. 5487-5492.
[56] S. T. Shi, K. J. Lee, H. Aizaki, S. B. Hwang and M. M. Lai, Hepatitis C virus RNA replication occurs on a detergent-resistant membrane that cofractionates with caveolin-2, J. Virol. 77 (2003), pp. 4160-4168.
[57] D. E. Goll, V. F. Thompson, H. Li, W. Wei and J. Cong, The calpain system, Physiol. Rev. 83 (2003), pp. 731-801.
[58] G. Waris and A. Siddiqui, Hepatitis C virus stimulates the expression of cyclooxygenase-2 via oxidative stress: role of prostaglandin E2 in RNA replication, J. Virol. 79 (2005), pp. 9725-9734.
[59] W. S. Ko, C. H. Guo, M. S. Yeh, L. Y. Lin, G. S. Hsu, P. C. Chen, M. C. Luo and C. Y. Lin, Blood micronutrient, oxidative stress, and viral load in patients with chronic hepatitis C, World J. Gastroenterol. 11 (2005), pp. 4697-4702.
[60] A. Melhem, M. Stern, O, Shibolet, E. Israeli, Z. Ackerman, O. Pappo, N. Hemed, M. Rowe, H. Ohana, G. Zabrecky, R. Cohen and Y. Ilan, Treatment of chronic hepatitis C virus infection via antioxidants: results of a phase I clinical trial, J. Clin. Gastroenterol. 39 (2005), pp. 737-742.
[61] Choi J, Xu Z, Ou J H. Triple decoding of hepatitis C virus RNA by programmed translational frameshifting. Mol Cell Biol 2003; 23:1489-1497.
[62] Varaklioti A, Vassilaki N, Georgopoulou U, Mavromara P. Alternate translation occurs within the core coding region of the hepatitis C viral genome. J Biol Chem 2002; 277:17713-17721.
[63] Walewski J L, Keller T R, Stump D D, Branch A D. Evidence for a new hepatitis C virus antigen encoded in an overlapping reading frame. Rna 2001; 7:710-721.
[64] Xu Z, Choi J, Yen T S, Lu W, Strohecker A, Govindarajan S, Chien D, et al. Synthesis of a novel hepatitis C virus protein by ribosomal frameshift. Embo J 2001; 20:3840-3848.
[65] Lindenbach B D, Rice C M. Unravelling hepatitis C virus replication from genome to function. Nature 2005; 436:933-938.
[66] Moradpour D, Penin F, Rice C M. Replication of hepatitis C virus. Nat Rev Microbiol 2007; 5:453-463.
[67] Choi J, Ou J H. Mechanisms of liver injury. III. Oxidative stress in the pathogenesis of hepatitis C virus. Am J Physiol Gastrointest Liver Physiol 2006; 290:G847-851.
[68] Choi J, Lee K J, Zheng Y, Yamaga A K, Lai M M C, Ou J-H. Reactive oxygen species suppress hepatitis C virus RNA replication in human hepatoma cells. Hepatology 2004; 39:81-89.
[69] Choi J, Forman H J, Ou J H, Lai M M, Seronello S, Nandipati A. Redox modulation of the hepatitis C virus replication complex is calcium dependent. Free Radic Biol Med 2006; 41:1488-1498.
[70] Blight K J, Kolykhalov A A, Rice C M. Efficient initiation of HCV RNA replication in cell culture. Science 2000; 290:1972-1974.
[71] Lohmann V, Korner F, Koch J, Herian U, Theilmann L, Bartenschlager R. Replication of subgenomic hepatitis C virus RNAs in a hepatoma cell line. Science 1999; 285:110-113.
[72] Simmonds P, Bukh J, Combet C, Deleage G, Enomoto N, Feinstone S, Halfon P, et al. Consensus proposals for a unified system of nomenclature of hepatitis C virus genotypes. Hepatology 2005; 42:962-973.
[73] Wakita T, Pietschmann T, Kato T, Date T, Miyamoto M, Zhao Z, Murthy K, et al. Production of infectious hepatitis C virus in tissue culture from a cloned viral genome. Nat Med 2005; 11:791-796.
[74] Heller T, Saito S, Auerbach J, Williams T, Moreen T R, Jazwinski A, Cruz B, et al. An in vitro model of hepatitis C virion production. Proc Natl Acad Sci USA 2005; 102:2579-2583.
[75] Kato T, Matsumura T, Heller T, Saito S, Sapp R K, Murthy K, Wakita T, et al. Production of infectious hepatitis C virus of various genotypes in cell cultures. J Virol 2007; 81:4405-4411.
[76] Seronello S, Sheikh M Y, Choi J. Redox regulation of hepatitis C in nonalcoholic and alcoholic liver. Free Radic Biol Med 2007; 43:869-882.

What is claimed is:

1. A method for inhibiting replication of the genome of an RNA virus in a host cell, comprising contacting the host cell with an effective amount of an agent that produces a subtoxic concentration of hydrogen peroxide, thereby inhibiting replication in the host cell.

2. The method of claim 1, wherein the agent is from the group enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)-a redox cycling quinine, 2,3 dimethoxy-1,4-naphthoquinone (DMNQ)-a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

3. The method of claim 1, wherein the agent is BSO.

4. The method of claim 1, wherein the agent is a combination of an effective amount of hydrogen peroxide and L-buthionine S,R-sufloximine (BSO).

5. The method of claim 1, wherein the agent is hydrogen peroxide.

6. The method of claim 6, further comprising contacting the host cell with an effective amount of BSO.

7. The method of claim 1, wherein the agent is a combination of glucose and glucose oxidase.

8. The method of claim 1, wherein the RNA virus is a Flavivirus.

9. The method of claim 9, wherein the Flavivirus is Hepatitis C Virus (HCV).

10. The method of claim 1, wherein viral replication comprises subgenomic viral replication without virus production.

11. The method of claim 1, wherein viral replication comprises inhibition of the complete retroviral replication cycle.

12. A method for inhibiting replication of the genome of an RNA virus in a subject in need thereof comprising administering an effective amount of an agent that produces a subtoxic concentration of hydrogen peroxide to the subject, thereby inhibiting replication.

13. A method for inhibiting replication of an RNA virus in a subject in need thereof, comprising administering an effective amount of an agent from the group of agent that facilitates enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)-a redox cycling quinine, 2,3 dimethoxy-1,4-naphthoquinone (DMNQ)-a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

14. The method of claim 13, wherein the agent is BSO.

15. The method of claim 13, wherein the agent is a combination of an effective amount of hydrogen peroxide and L-buthionine S,R-sufloximine (BSO).

16. The method of claim 13, wherein the agent is hydrogen peroxide.

17. The method of claim 18, further comprising contacting the host cell with an effective amount of BSO.

18. The method of claim 13, wherein the agent is a combination of glucose and glucose oxidase.

19. The method of claim 13, wherein the RNA virus is a Flavivirus.

20. The method of claim 19, wherein the Flavivirus is Hepatitis C Virus (HCV).

21. The method of claim 13, wherein viral replication comprises subgenomic viral replication without virus production.

22. The method of claim 13, wherein viral replication comprises inhibition of the complete viral replication cycle.

23. A method for treating liver disease incident to Hepatitis C Viral infection in a subject, comprising administering to the subject an effective amount of an agent that produces an anti-viral amount of reactive oxygen species in the subject.

24. A method for treating liver disease incident to HCV infection in a subject in need thereof, comprising administering to the subject an effective amount of an agent from the group enzymatic generation with glucose oxidase and glucose, L-buthionine S,R-sulfoximine (BSO) or other agent that decreases antioxidant defense of the cell and therefore amplifies the effects of the endogenously generated reactive oxygen species, tTert-butylhydroquinone (TBHQ)-a redox cycling quinine, 2,3 dimethoxy-1,4-naphthoquinone (DMNQ)-a redox cycling quinine, ascorbate, dehydroascorbate, agents that induce and/or activate NAD(P)H oxidase family proteins (Nox proteins), BCNU or other inhibitor of glutathione reductase that decreases antioxidant defense of the cell and therefore amplifies the effects of the endogenously generated oxidants, menadione or other redox cycling quinine, diazyquone (AZQ), adriamycin, 2,5-diaziridinyl-1,4-benzoquinone (DZQ) or other quinone anticancer agents or butylated hydroxyanisole (BHA) that produces TBHQ.

25. The method of claim 24, wherein the agent is BSO.

26. The method of claim 24, wherein the agent is a combination of an effective amount of hydrogen peroxide and L-buthionine S,R-sufloximine (BSO).

27. The method of claim 24, wherein the agent is hydrogen peroxide.

28. The method of claim 24, further comprising contacting the host cell with an effective amount of BSO.

29. The method of claim 28, wherein the agent is a combination of glucose and glucose oxidase.


SEQUENCE LISTING (3 sequences)

SEQ ID NO: 1 - DNA, 19 bp, Artificial Sequence
  Description of Artificial Sequence Synthetic primer
tctgcggaaccggtgagta

SEQ ID NO: 2 - DNA, 19 bp, Artificial Sequence
  Description of Artificial Sequence Synthetic primer
tcaggcagtaccacaaggc

SEQ ID NO: 3 - DNA, 21 bp, Artificial Sequence
  Description of Artificial Sequence Synthetic probe
ccagtcttcccggcaattccg